(19) United States
(12) Patent Application Publication
(10) Pub. No.: US 2026/0116922 A1
(43) Pub. Date: Apr. 30, 2026

(54) HIGHLY STABLE OXYTOCIN DERIVATIVES

(71) Applicant: UNIVERSITAET WIEN, Vienna (AT)

(72) Inventors: Thomas KREMSMAYR, Vienna (AT); Markus MUTTENTHALER, Vienna (AT)

(73) Assignee: UNIVERSITAET WIEN, Vienna (AT)

(21) Appl. No.: 18/715,925

(22) PCT Filed: Dec. 21, 2022
(86) PCT No.: PCT/EP2022/087248

§ 371 Date: Jun. 3, 2024

(30) Foreign Application Priority Data
Dec. 21, 2021 (EP) 21216288.7

(51) Int. Cl. C07K 7/64; A61K 38/00

(52) U.S. Cl. CPC C07K 7/64; A61K 38/00

(57) ABSTRACT

The present invention relates to novel compounds of general formula (I), (I), wherein Z¹, X¹, R², R²¹, n1, W, R³, R³¹, n2, X², R^{x}, R⁷, R⁷¹, R⁸, Y, n4, n8, R⁸¹, n3, X³, R^{y}, X⁴, R⁹, Z⁹, and m have the meanings given in the description and claims, process for preparing these compounds, and their use as medicaments, specifically for treating, preventing, or ameliorating gastrointestinal disorders.

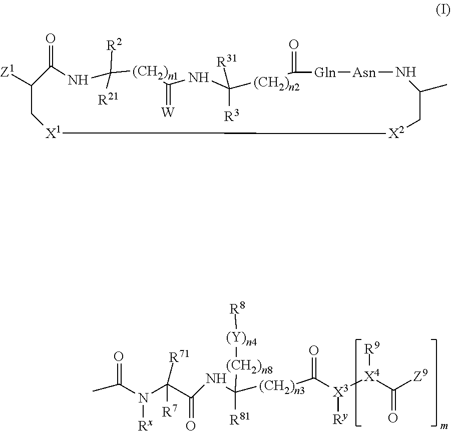

### CROSS-REFERENCE TO RELATED APPLICATIONS

This application is a National Phase Application under 35 U.S.C § 371 of International Application PCT/EP2022/087248, filed Dec. 21, 2022, which claims the benefit of European Patent Application No. 21216288.7, filed Dec. 21, 2021, the entireties of which are incorporated herein by reference.

### REFERENCE TO A SEQUENCE LISTING

This application contains a Sequence Listing XML, which has been submitted electronically and is hereby incorporated by reference in its entirety. Said Sequence Listing XML, created on Nov. 25, 2024, is named 241125_UW010PUS_Sequence_Listing_corr.xml and is 2,335 bytes in size.

### DESCRIPTION

This project has received funding from the European Research Council (ERC) under the European Union's Horizon 2020 research and innovation programme (grant agreement No 714366) and the Austrian Academy of Sciences (DOC fellowship 25139).

### FIELD OF THE INVENTION

The present invention relates to novel oxytocin (OT) derivatives of the general formula I

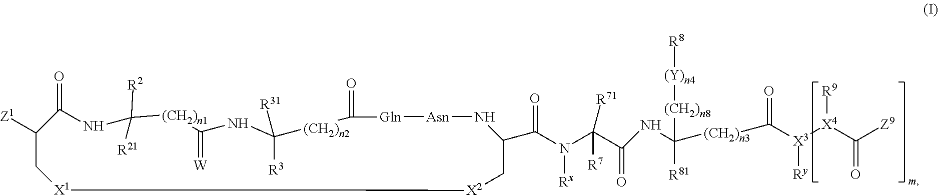

wherein Z¹, X¹, R⁷, R⁷¹, n1, W, R³, R³¹, n2, X², R^{x}, R⁷, R⁷¹, Y, n4, n8, R⁵¹, n3, X³, R^{y}, X⁴, R⁹, Z⁹, and m have the meanings given in the description and claims, process for preparing these compounds, and their use as medicaments, specifically for treating, preventing, or ameliorating gastrointestinal disorders.

### BACKGROUND ART

Oxytocin (OT) is a neurohypophysial peptide that acts via the OT receptor (OTR), a G protein-coupled receptor (GPCR) belonging to the rhodopsin-like/class A family. OT/OTR signaling regulates fundamental physiological functions in the central and peripheral nervous system. Centrally, OT is involved in the formation of complex social behavior, maternal care, stress and anxiety. In the periphery, OT plays a key role in reproduction, including childbirth (contraction of uterine smooth muscles), lactation for breastfeeding, and ejaculation.

OT/OTR signaling also occurs in the enteric nervous system. Increasing evidence suggests that OT/OTR function is physiologically involved in the development and maintenance of the gastrointestinal system, including gut motility, sensation, inflammation, and epithelial gut-barrier function.

Clinically, exogenous OT has been used as an intravenous drug to induce labor and treat postpartum hemorrhage, and intranasally to elicit lactation. Based on its multifunctional physiological roles, the OT/OTR system has emerged as a potential drug target in various conditions beyond current clinal use, including neuropsychiatric disorders (e.g. autism, schizophrenia, anxiety, depression, addiction) cardiovascular conditions, cancer, inflammation and pain.

Emerging evidence suggests that the therapeutic potential of OT/OTR in pain and inflammatory conditions together with its physiological relevance for gut functions presents a handle for the treatment of gastrointestinal disorders such as Inflammatory Bowel Diseases (IBD, including ulcerative colitis and Crohn's disease) and Irritable Bowel Syndrome (IBS). Systemic administration of OT triggers analgesia under visceral pain conditions, alleviates intestinal inflammation and promotes gut-barrier function. Moreover, OT acts locally in the gut by reducing pain-associated neuronal signaling under conditions of abdominal diseases.

Compounds that target luminally accessible receptors locally in the gut (via oral or rectal route) present a gut-specific treatment approach that circumvents adverse global side-effects frequently encountered with systemic drug administration (e.g., injectables) and removes the necessity of crossing the gut absorption barrier.

However, the digestive environment of the gut is a major hurdle for the development of orally administrable peptide drugs and is a substantially harsher and more complex enzymatic environment compared to serum/plasma. In the stomach (experimentally reflected via simulated gastric fluid, SGF), the digestive enzyme pepsin (endopeptidase, cleaves peptide bonds at the site of aromatic and hydrophobic amino acids) initiates the digestion process under strongly acidic conditions (hydrochloric acid, pH ˜1.2). In the intestine, a mixture of peptidases, lipases and amylases (pancreatic enzymes) presents a serious stability hurdle for peptide drugs (experimentally typically reflected via simulated intestinal fluid, SIF). Pancreatic peptidases span a broad substrate specificity and include the endopeptidases trypsin (cleavage sites: Arg, Lys), chymotrypsin (cleavage sites: aromatic, hydrophobic residues) and elastase (cleavage sites: small hydrophobic residues) as well as the exopeptidases carboxypeptidase A (cleavage sites: aromatic, neutral, acidic amino acids) and B (cleavage sites: Arg, Lys).

Chemically, OT is a nonapeptide with a highly conserved structural arrangement which includes a six-amino-acid-containing macrocycle (single disulfide bond) and a three-residue C-terminal tail sequence. OT is stable in the stomach but rapidly degraded by pancreatic peptidases in the intestine, limiting its therapeutic potential in gastrointestinal disorders [1-3].

Recently, OT derivatives comprising a fatty acid moiety conjugated to a mutated Cys⁸ residue have been described in WO2021/126990A1. These OT derivatives are OTR agonists with improved blood circulation half-lives based on binding to serum albumin.

In WO2011/035330A2, OT derivatives comprising chemically modified amino acids that can still activate OTR have been disclosed.

Fragiadaki M. et al. (2007) describe the synthesis and biological activity of oxytocin analogues containing conformationally restricted residues in position 7 [10].

Flouret G. et al. (2003) describe analogues of oxytocin antagonists with truncated C-terminus or modified amino acid side chain in position 8 [11].

Parmar A. (2019) describe an OTR-specific PET tracer having a modification in position 8 of native oxytocin consisting of a lysine residue labelled with [18F] SFB [12].

Muttenthaler M. et al. (2010) describe the disulfide bond engineering of oxytocin [13].

Dongren R. et al. (2015) describe oxytocin variants with various amino acids in position 8, including Pro8, Ala8, Thr8, and Phe8 [14].

However, OT derivatives explicitly providing improved gut stability have not been developed so far. OT displays a very complex structure-activity relationship and stabilizing modifications easily can cause inactivation. Gut-stable compounds capable of targeting OTR locally in the gut (via oral or rectal route) present an attractive gut-specific treatment option and remove the necessity of crossing the gastrointestinal epithelial barrier, a major hurdle in the development of oral peptide drugs.

Thus, there is an unmet need for gut-stable compounds that target OTR locally in the gut. Such compounds would pave the way to a new generation of stable gut-specific peptide therapeutics and molecular probes targeting OTR in gastrointestinal disorders.

### SUMMARY OF THE INVENTION

It is the object of the present invention to provide potent and gut-stable OT derivatives capable of locally targeting OTR in the gut.

The object is solved by the subject matter of the present invention.

The present invention provides OT derivatives with improved gut stability capable to act as OTR agonists or as OTR antagonists. A range of chemical modifications to prevent molecular degradation of OT in the gut are provided in the present invention and surprisingly led to a series of compounds that are gut-stable and active at the OTR.

According to the invention there is provided a compound of general formula I

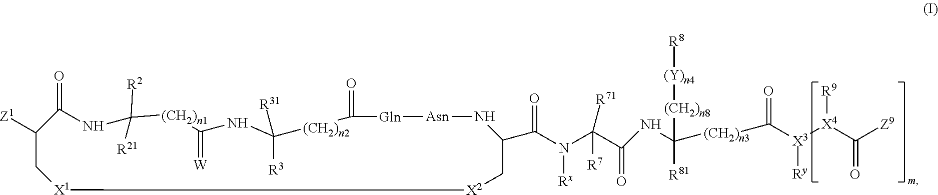

wherein
Z¹ (N-terminus) denotes NH₂, H or OH;
Z⁹ (C-terminus) denotes NH₂, or OH; or
Z⁹ denotes NH if m denotes 2, 3, 4, 5, or 6;
X¹ and X² independently from one another denote S, Se, or CH₂;
R²¹, R³¹, R⁷¹, and R⁸¹ independently from one another denote H, C₁₋₆alkyl, halogen, or CF₃;
R^{x} denotes H or a group, optionally substituted by C₆₋₁₀ aryl and/or optionally substituted by one or more identical or different R^{a} and/or R^{b}, selected from among C₁₋₆alkyl, C₂₋₆alkenyl, or C₂₋₆alkynyl;
R^{y} denotes H or a group, optionally substituted by C₆₋₁₀ aryl and/or optionally substituted by one or more identical or different R^{a} and/or R^{b}, selected from C₁₋₆alkyl, C₂₋₆alkenyl, or C₂₋₆alkynyl;
n1 denotes 0, 1, 2, or 3, and wherein n1 denotes 0 if R²¹ is different from H;
n2 denotes 0, 1, 2, or 3, and wherein n2 denotes 0 if R³¹ is different from H;
n3 denotes 0, 1, 2, or 3, and wherein n3 denotes 0 if R⁸¹ is different from H;
n4 denotes 0 or 1;
n8 denotes 0, 1, 2, 3, 4, 5, 6, 7, 8, 9, or 10;
m denotes 0, 1, 2, 3, 4, 5, or 6;
W denotes O or S;
X³ denotes N; or
X³ denotes 0 or NH if m denotes 0;
X⁴ denotes CH or N;
Y denotes NH or O;
R² denotes Tyr together with the peptide backbone of residue 2; or
R² denotes H or a group, optionally substituted by one or more identical or different R^{a} and/or R^{b}, selected independently from among C₁₋₁₀alkyl, C₂₋₁₀ alkenyl, C₂₋₁₀alkynyl, C₃₋₈cycloalkyl, C₄₋₈cycloalkylalkyl, C₆₋₈ aryl, C₇₋₈ arylalkyl, 3-8 membered heterocycloalkyl, 4-8 membered heterocycloalkylalkyl, 5-8 membered heteroaryl, and 6-8 membered heteroarylalkyl;
R³ denotes lie together with the peptide backbone of residue 3; or
R³ denotes H or a group, optionally substituted by one or more identical or different R^{a} and/or R^{b}, selected independently from among C₁₋₁₀ alkyl, C₂₋₁₀ alkenyl, C₂₋₁₀alkynyl, C₃₋₈ cycloalkyl, C₄₋₈cycloalkylalkyl, C₆₋₈ aryl, C₇₋₈ arylalkyl, 3-8 membered heterocycloalkyl, 4-8 membered heterocycloalkylalkyl, 5-8 membered heteroaryl, and 6-8 membered heteroarylalkyl;
R⁷ denotes H or a group, optionally substituted by one or more identical or different R^{a} and/or R^{b}, selected independently from among C₁₋₁₀ alkyl, C₂₋₁₀ alkenyl, C₂₋₁₀alkynyl, C₃₋₈cycloalkyl, C₄₋₈cycloalkylalkyl, C₆₋₈ aryl, C₇₋₈ arylalkyl, 3-8 membered heterocycloalkyl, 4-8 membered heterocycloalkylalkyl, 5-8 membered heteroaryl, and 6-8 membered heteroarylalkyl; or
R⁷ together with R^{x} forms an optionally substituted 3 to 6 membered heterocycloalkyl ring;
R⁸ denotes —C(O)R^{c}, —C(R^{c})=NOR^{c}, or a group, optionally substituted by one or more identical or different R^{a} and/or R^{b}, selected independently from among C₁₋₁₀alkyl, C₂₋₁₀ alkenyl, C₂₋₁₀ alkynyl, 3-13 membered heterocycloalkyl, and 5-13 membered heteroaryl, and wherein R⁸ optionally comprises a terminal lipidation and/or PEGylation, and/or labeling;
R⁹ denotes H or C₁₋₅alkyl;
each R^{a} independently of one another denotes H or a group, optionally substituted by one or more identical or different R^{b} and/or R^{c}, selected from among C₁₋₆alkyl, C₂₋₆alkenyl, C₂₋₆alkynyl, C₃₋₈cycloalkyl, C₄₋₈cycloalkylalkyl, C₆₋₈ aryl, C₇₋₈ arylalkyl, 3-8 membered heterocycloalkyl, 4-8 membered heterocycloalkylalkyl, 5-8 membered heteroaryl and 6-8 membered heteroarylalkyl;
each R^{b} is a suitable substituent and is selected in each case independently of one another from among ═O, —OR, C₁₋₃haloalkyloxy, —OCF₃, ═S, —SR^{c}, =NR^{c}, =NOR^{c}, —NR^{c}R^{c}, —ONR^{c}R^{c}, —N(OR^{c})R^{c}, —N(R^{g})NR^{c}R^{c}, halogen, —CF₃, —CN, —NC, —OCN, —SCN, —NO, —NO₂, =N₂, —N₃, —S(O)R^{c}, —S(O)OR^{c}, —S(O)₂R^{c}, —S(O)₂OR^{c}, —S(O)NR^{c}R^{c}, —S(O)₂NR^{c}R^{c}, —OS(O)R^{c}, —OS(O)₂R^{c}, —OS(O)₂OR^{c}, —OS(O)NR^{c}R^{c}, —OS(O)₂NR^{c}R^{c}, —C(O)R^{c}, —C(O)OR^{c}, —C(O)SR^{c}, —C(O)NR^{c}R^{c}, —C(O)N(R^{g})NR^{c}R^{c}, —C(O)N(R^{g})OR^{c}, —C(NR^{h})NR^{c}R^{c}, —C(NOH)R^{c}, —C(NOH)NR^{c}R^{c}, —OC(O)R^{c}, —OC(O)OR^{c}, —OC(O)SR^{c}, —OC(O)NR^{c}R^{c}, —OC(NR⁹)NR^{c}R^{c}, —SC(O)R^{c}, —SC(O)OR^{c}, —SC(O)NR^{c}R^{c}, —SC(NR⁹)NR^{c}R^{c}, —N(R^{g})C(O)R^{c}, and —N[C(O)R]₂;
each R^{c} independently of one another denotes H or a group, optionally substituted by one or more identical or different R^{d} and/or R^{c}, selected from among C₁₋₆alkyl, C₂₋₆alkenyl, C₂₋₆alkynyl, C₃₋₈cycloalkyl, C₄₋₈cycloalkylalkyl, C₆₋₈ aryl, C₇₋₈ arylalkyl, 3-8 membered heterocycloalkyl, 4-8 membered heterocycloalkylalkyl, 5-8 membered heteroaryl and 6-8 membered heteroarylalkyl;
each R^{d} denotes a suitable substituent and is selected in each case independently of one another from among ═O, —OR^{e}, C₁₋₃haloalkyloxy, —OCF₃, ═S, —SR^{e}, =NR^{e}, =NOR^{e}, —NR^{e}R^{e}, —ONR^{e}R^{e}, —N(OR^{e})R^{e}, —N(R^{g})NR^{e}R^{e}, halogen, —CF₃, —CN, —NC, —OCN, —SCN, —NO, —NO₂, =N₂, —N₃, —S(O)R^{e}, —S(O)OR^{e}, —S(O)₂R^{e}, —S(O)₂OR^{e}, —S(O)NR^{e}R^{e}, —S(O)₂NR^{e}R^{e}, —OS(O)R^{e}, —OS(O)₂R^{e}, —OS(O)₂OR^{e}, —OS(O)NR^{e}R^{e}, —OS(O)₂NR^{e}R^{e}, —C(O)R^{e}, —C(O)OR^{e}, —C(O)SR^{e}, —C(O)NR^{e}R^{e}, —C(O)N(R^{e})NR^{e}R^{e}, —C(O) N(R^{g})OR^{e}, —C(NR^{g})NR^{e}R^{e}, —C(NOH)R^{e}, —C(NOH)NR^{e}R^{e}, —OC(o)R^{e}, —OC(O)OR^{e}, —OC(O)SR^{e}, —OC(O)NR^{e}R^{e}, —OC(NR^{g})NR^{e}R^{e}, —SC(O)R^{e}, —SC(O)OR^{e}, —SC(O)NR^{e}R^{e}, —SC(NR⁹)NR^{e}R^{e}, —N(R^{g})C(O)R^{e}, and —N[C(O)R^{e}]₂; and
each R^{e} independently of one another denotes H or a group selected from among C₁₋₆alkyl, C₂₋₆alkenyl, C₂₋₆alkynyl, C₃₋₈cycloalkyl, C₆₋₈ aryl, 3-8 membered heterocycloalkyl, and 5-8 membered heteroaryl; and
each R^{g} independently of one another denotes H, C₁₋₆alkyl, C₂₋₆alkenyl, C₂₋₆alkynyl, C₃₋₈cycloalkyl, C₆₋₈ aryl, 3-8 membered heterocycloalkyl, or 5-8 membered heteroaryl;
with the proviso that R⁸, n4, n8, and Y together with the peptide backbone of residue 8 is different from Leu, lie, cyclopentyl-Gly (Cpg), Lys, Arg, or the enantiomers thereof;
with the proviso that if R^{g} comprises a terminal lipidation and/or PEGylation and/or labeling, R² together with the peptide backbone of residue 2 is different from Tyr, and/or R³ together with the peptide backbone of residue 3 is different from Ile, and/or one of X¹ or X² is different from S; and
with the proviso that if R^{g}, n4, n8, and Y together with the peptide backbone of residue 8 is Ala or Thr, then R² together with the peptide backbone of residue 2 is different from Tyr, and/or R³ together with the peptide backbone of residue 3 is different from Ile, and/or one of X¹ or X² is different from S;
optionally in the form of the tautomers, the racemates, the enantiomers, the diastereomers, hydrates, and mixtures thereof, and optionally the pharmacologically acceptable salts thereof.

Specifically, R⁸ is —C(O)R^{c}, —C(R^{c})=NOR^{c}, or a group, optionally substituted by one or more identical or different R^{a} and/or R^{b}, selected independently from among
C₁₋₁₀alkyl, C₂₋₁₀ alkenyl, C₂₋₁₀ alkynyl, 3-13 membered heterocycloalkyl, or
5-13 membered heteroaryl, wherein R^{b} is preferably selected from among ═O, —OR^{c}, C₁₋₃haloalkyloxy, —OCF₃, —NR^{c}R^{c}, —N(OR^{c})R^{c}, halogen, —CF₃, —C(O)R^{c}, —C(O)OR^{c}, —C(O)NR^{c}R^{c}, —C(O)N(R⁹)NR^{c}R^{c}, —C(O)N(R⁹)OR, —C(NR⁹)NR^{c}R^{c}, and —N(R^{g})C(O)R^{c}.

Specifically, the terminal lipidation and/or PEGylation of R⁸ comprises a group selected from the group consisting of polyethylene glycol (PEG), PEG substituted with one or more amino acids and/or one or more fatty acids, and (PEG substituted with one amino acid)₁₋₅ optionally substituted with one or more fatty acid.

More specifically, in the terminal PEGylation of R⁸, PEG comprises PEG₂₋₅₀.

More specifically, in the terminal lipidation and/or PEGylation of R⁸, the amino acids are selected from Lys and Glu optionally in the form of the β-amino acids, the γ-amino acids, the enantiomers, and the salts thereof.

More specifically, in the terminal lipidation and/or PEGylation of R⁸, the fatty acid is C₆₋₃₀ or C₆₋₃₀ fatty diacid.

Specifically, the terminal labeling of R⁸ comprises a group selected from the group consisting of biotin; fluorophores; metal chelators, preferably DOTA, NODA, and NODAGA; antigens; MS-, NMR-, PET- and MRI-active tags; technical recognition tags; and biological recognition tags.

Specifically, R² denotes Tyr together with the peptide backbone of residue 2, or a C₁₋₁₀alkyl group optionally substituted by R^{a}, preferably by C₆₋₈ aryl, C₇₋₈ arylalkyl, 5-8 membered heteroaryl, and 6-8 membered heteroarylalkyl, optionally further substituted by one or more identical or different R^{b} and/or R^{c}, preferably by
C₁₋₆alkyl, ═O, —OR^{c}, C₁₋₃haloalkyloxy, —OCF₃, —NR^{c}R^{c}, —N(OR^{c})R^{c}, halogen, —CF₃, —C(O)R^{c}, —C(O)OR^{c}, —C(O)NR^{c}R^{c}, —C(O)N(R^{g})NR^{c}R^{c}, —C(O)N(R^{g})OR^{c}, —C(NR^{g})NR^{c}R^{c}, or —N(R^{g})C(O)R^{c}.

Specifically, R³ denotes lie together with the peptide backbone of residue 3, or a group, selected independently from among C₁₋₁₀ alkyl, C₃₋₈ cycloalkyl, optionally substituted by one or more identical or different R^{a} and/or R^{b}, wherein R^{b} is preferably selected from among ═O, —OR^{c}, C₁₋₃haloalkyloxy, —OCF₃, —NR^{c}R^{c}, —N(OR^{c})R^{c}, halogen, —CF₃, —C(O)R^{c}, —C(O)OR^{c}, —C(O)NR^{c}R^{c}, —C(O)N(R^{g})NR^{c}R^{c}, —C(O)N(R^{g})OR^{c}, —C(NR⁹)NR^{c}R^{c}, and —N(R^{g})C(O)R^{c}.

Specifically, R⁷ denotes H or C₁₋₁₀ alkyl optionally substituted by one or more identical or different R^{a} and/or R^{b}, wherein R^{b} is preferably selected from among ═O, —OR^{c}, C₁₋₃haloalkyloxy, —OCF₃, —NR^{c}R^{c}, —N(OR^{c})R^{c}, halogen, —CF₃, —C(O)R^{c}, —C(O)OR^{c}, —C(O)NR^{c}R^{c}, —C(O)N(R⁹)NR^{c}R^{c}, —C(O)N(R^{g})OR^{c}, —C(NR⁹)NR^{c}R^{c}, and —N(R^{g})C(O)R^{c}, or
R⁷ together with R^{x} forms an optionally substituted 3 to 5 membered heterocycloalkyl ring, preferably pyrrolidine, thiazolidine, or hydroxypyrrolidine.

Specifically, R²¹, R³¹, R⁷¹, and R⁸ independently from one another denote H, C₁₋₆alkyl, halogen, or CF₃.

Specifically, R²¹ denotes H or methyl.

Specifically, R³¹ denotes H or methyl.

Specifically, R⁷¹ denotes H.

Specifically, R⁸¹ denotes H or methyl.

Specifically, R² denotes C₁₋₃alkyl group substituted by optionally substituted C₆₋₈ aryl.

Specifically, said C₆₋₈ aryl is substituted by one or more identical moieties selected from the group consisting of C₁₋₆alkyl, ═O, —OR, C₁₋₃haloalkyloxy, —OCF₃, —NR^{c}R^{c}, —N(OR^{c})R^{c}, halogen, —CF₃, —C(O)R^{c}, —C(O)OR^{c}, —C(O)NR^{c}R^{c}, —C(O)N(R′)NR^{c}R^{c}, —C(O)N(R^{g})OR, —C(NR^{g})NR^{c}R^{c}, or —N(R^{g})C(O)R^{c}.

Specifically, R³ denotes lie together with the peptide backbone of residue 3.

Specifically, R³ denotes optionally substituted C₁₋₁₀alkyl or C₅₋₆cycloalkyl.

Specifically, R³ denotes H or
R⁷ together with R^{x} forms an optionally substituted 3 to 5 membered heterocycloalkyl ring.

Specifically, the 3 to 5 membered heterocycloalkyl ring is selected from the group consisting of pyrrolidine, thiazolidine, and hydroxypyrrolidine.

Specifically, R^{x} denotes H or optionally substituted C₁₋₆alkyl.

Specifically, Z⁹, R^{y}, m, X³, X⁴, and R⁹ together form Gly-NH₂.

Specifically, the compounds of general formula I are represented by formula VI,

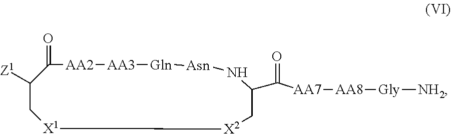

wherein
Z¹ denotes NH₂ or H;
X¹ and X² independently from one another denote S or CH₂;
AA2 denotes Tyr, Tyr(Me), (C_{α}-Me)Tyr, (C_{α}-Me)Phe, Phe(4-Me), Phe(4-CF₃), or Phe(4-F);
AA3 denotes lie, Cpg, (C_{α}-Me)Val, or Chg;
AA7 denotes Pro, (4-FBzl)Gly, thioPro, or Hyp;
AA8 denotes Lys(Ac), Dpr(Ac), (C_{α}-Me)Leu, (β³-homo)Leu, Dpr(Piv), or Lys(γ Glu, α Palm).

According to the invention there is further provided the compound of general formula I, optionally in the form of the tautomer, the racemates, the enantiomers, the diastereoisomers, hydrates, and mixtures thereof, or the pharmaceutically acceptable salt thereof for use as a medicament.

According to the invention there is further provided the compound of general formula I, optionally in the form of the tautomers, the racemates, the enantiomers, the diastereoisomers, hydrates, and mixtures thereof, or the pharmaceutically acceptable salts thereof for use in the treatment or prevention of gastrointestinal disorders including symptoms and/or causes associated with inflammatory bowel disease (IBD) including ulcerative colitis and Crohn's disease and symptoms and/or causes associated with irritable bowel syndrome (IBS), wherein said symptoms and/or causes are abdominal/visceral pain, constipation, diarrhea, abnormal bowel movement, inflammation and rectal bleeding.

According to the invention there is further provided a pharmaceutical preparation containing as active substance one or more compounds of general formula I, optionally in the form of the tautomers, the racemates, the enantiomers, the diastereoisomers, hydrates, and mixtures thereof, or the pharmaceutically acceptable salts thereof, optionally in combination with conventional excipients and/or carriers.

According to the invention there is further provided a pharmaceutical preparation comprising a compound of general formula I, optionally in the form of the tautomers, the racemates, the enantiomers, the diastereoisomers, hydrates, and mixtures thereof, or also pharmaceutically acceptable salts of all the above-mentioned forms, and at least one further active substance different from formula I.

### BRIEF DESCRIPTION OF DRAWINGS

FIG. 1: Chemical structure and sequence (SEQ ID NO: 1) of oxytocin (OT)

FIG. 2: Metabolic cleavage sites of native oxytocin (OT) in the intestine.

### DESCRIPTION OF EMBODIMENTS

One embodiment of the invention relates to compounds of the general formula I, wherein said compound is a peptide, more specifically a derivative of the peptide OT.

The term “derivative” refers to the OT molecule which has site-selective chemical modifications according to the general formula I.

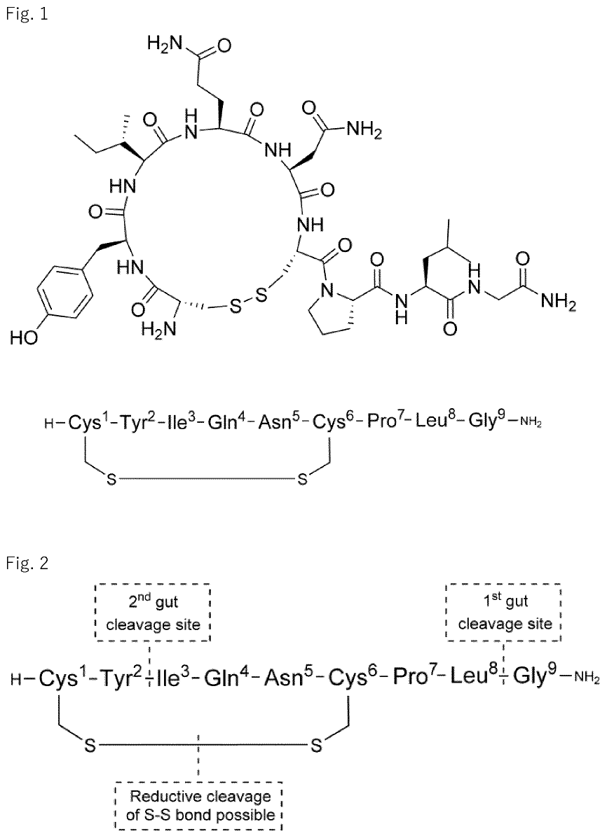

The chemical sequence of native bioactive OT is displayed in FIG. 1 and given in SEQ ID NO: 1. FIG. 1 highlights the numbering of the amino acids in the sequence, according to which the numbering of residues is referred to as herein. For example, the residue corresponding to Cys¹ in OT is referred to as residue 1, the residue corresponding to Tyr² in OT is referred to as residue 2 etc. As an alternative to the term “residue”, the term “position” is used herein to refer to a residue corresponding to the respective residue in OT or in an OT derivative of the invention.

The metabolic degradation of OT in the intestine (simulated intestinal fluid, SIF) is depicted in FIG. 2. OT is stable in the stomach (t_{1/2}^{SGF}>24 h) but rapidly degraded by pancreatic peptidases in the intestine (t_{1/2}^{SIF} 8±1 min). The metabolism of OT in intestinal fluid proceeds via an initial stepwise cleavage of peptide bonds at the C-terminal tail (Leu⁸-Gly⁹ then Pro⁷-Leu⁸) (FIG. 2). The peptide bond between Tyr²-Ile³ is the second major cleavage site which is recognized by pancreatic chymotrypsin, causing inactivation by opening of the six-residue ring moiety (FIG. 2).

According to one embodiment of the invention, a potent and gut-stable OT derivative is provided herein by introducing site-selective chemical modifications that prevent the metabolic fate of OT in intestinal fluid. The OT derivative can act as an agonist or as an antagonist. As an agonist, the OT derivative has retained its capability to bind and activate OTR. As an antagonist, the OT derivative binds to the OTR but does not activate OTR and is thus able to block the activity of other OTR-activating compounds.

Peptides have a regularly repeating part and a variable part. The variable part of a peptide is formed by the distinctive side chains of the amino acids. The regularly repeating part of a peptide is the “peptide backbone” or simply “backbone” and is formed by the α-carbon of an amino acid, the atoms of an amino acid taking part in the peptide bond, i.e., the amino group and the carboxyl group of the amino acid, and possibly further atoms involved in peptide backbone modifications.

As used herein, amino acids refer to the 21 naturally occurring amino acids as well as to non-natural amino acids. Non-natural amino acids comprise modifications of the side chain and/or modifications of the peptide backbone. The naturally occurring amino acids are distinguished based on their distinct side chains which form the variable part in a peptide. These 21 naturally occurring amino acids are listed in the following together with their respective three-letter and single-letter code: Alanine (Ala, A), Asparagine (Asn, N), Cysteine (Cys, C), Glutamine (Gln, Q), Glycine (Gly, G), Isoleucine (lie, 1), Leucine (Leu, L), Methionine (Met, M), Phenylalanine (Phe, F), Proline (Pro, P), Serine (Ser, S), Threonine (Thr, T), Tryptophan (Trp, W), Tyrosine (Tyr, Y), Valine (Val, V), Histidine (His, H), Arginine (Arg, R), Lysine (Lys, K), Aspartic acid (Asp, D), Glutamic acid (Glu, E), and Selenocysteine (Sec, U).

Herein, the three-letter code is used for referring to an amino acid. For example, “Y” is used as a variable in e.g., formula I as defined herein, and “Tyr” is used for referring to the amino acid tyrosine.

Peptide backbone modifications are for example peptide backbone extension, carbonyl replacement, C α substitution, C α replacement, N replacement, N alkylation, and modifications of the C-terminus.

According to one embodiment of the invention, the chemical composition of residue 1 is defined in the general formula I. The variable X¹ defines possible modifications of the side chain of the amino acid of residue 1, variable Z¹ defines possible modifications of the peptide backbone.

According to one embodiment of the invention, the chemical composition of residue 2 is defined in the general formula I. The variable R² defines possible modifications of the side chain of the amino acid of residue 2. The variables R²¹, n1, and W define possible modifications of the peptide backbone. As an example, if R² denotes benzyl-OH, R²¹ denotes H, n1 denotes 0, and W denotes 0, the amino acid Tyr is formed at position 2 by R² together with the peptide backbone of residue 2.

According to one embodiment of the invention, the chemical composition of residue 3 is defined in the general formula I. The variable R³ defines possible modification of the side chain of the amino acid of residue 3. Variables R³¹ and n2 define possible modifications of the peptide backbone. As an example, if R³ denotes —CH(CH₃)CH₂CH₃, R³¹ denotes H, and n2 denotes 0, the amino acid Ile is formed by R³ together with the peptide backbone of residue 3.

According to one embodiment of the invention, the chemical composition of residue 6 is defined in general formula I. Variable X² defines possible modifications of the side chain of the amino acid of residue 6.

According to one embodiment of the invention, the chemical composition of residue 7 is defined in general formula I. Variable R⁷, or alternatively variables R¹ and R⁷, define possible modifications of the side chain of the amino acid of residue 7. Variables Rand R⁷¹ define possible modifications of the peptide backbone. Non-limiting examples of residue 7 according to the invention are Pro and thioproline (thioPro) if variables R^{x} and R⁷ together form the side chain.

According to one embodiment of the invention, the chemical composition of residue 8 is defined in general formula I. Variables n8, n4, Y, and R⁸ define possible modifications of the side chain of the amino acid of residue 8. Variables R⁸¹ and n3 define possible modifications of the peptide backbone.

According to a specific embodiment of the invention, the variables R⁸, n8, n4, and Y together with the peptide backbone and the optional terminal lipidation and/or PEGylation and/or labeling of residue 8 do not form the amino acids Leu, lie, Lys, Arg, Cpg, or the enantiomers thereof.

The term “Cpg” as used herein refers to cyclopentyl-Gly.

According to another specific embodiment of the invention, if R⁸ comprises a terminal lipidation and/or PEGylation and/or labeling, R² together with the peptide backbone of residue 2 is different from Tyr, and/or R³ together with the peptide backbone of residue 3 is different from Ile, and/or one of X¹ or X² is different from S.

More specifically, according to the invention, n8, n4, Y, and R⁸ together are different from —CH₂CH(CH₃)CH₃ if R⁸¹ denotes H, and if n3 denotes 0. Thus, the amino acid Leu is not formed by n8, n4, Y, and R^{a} together with the peptide backbone of residue 8 in the compound of the invention.

More specifically, according to the invention, n8, n4, Y, and R⁸ together are different from —CH(CH₃)CH₂CH₃ if R⁸¹ denotes H, and if n3 denotes 0. Thus, the amino acid Ile is not formed by n8, n4, Y, and R^{a} together with the peptide backbone of residue 8 in the compound of the invention.

More specifically, according to the invention, n8, n4, Y, and R⁸ together are different from —(CH₂)₄NH₃ or —(CH₂)₄NH₂ if R⁸¹ denotes H, and if n3 denotes 0. Thus, the amino acid Lys is not formed by n8, n4, Y, and R^{a} together with the peptide backbone of residue 8 in the compound of the invention.

More specifically, according to the invention, n8, n4, Y, and R^{a} together are different from —(CH₂)₃NHC(═NH₂)NH₂ or —(CH₂)₃NHC(═NH)NH₂ if R⁸¹ denotes H, and if n3 denotes 0. Thus, the amino acid Arg is not formed by n8, n4, Y, and R⁸ together with the peptide backbone of residue 8 in the compound of the invention.

More specifically, according to the invention, n8, n4, Y, and R⁸ together are different from -cyclopentyl if R⁸¹ denotes H, and if n3 denotes 0. Thus, cyclopentyl-Gly is not formed by n8, n4, Y, and R^{a} together with the peptide backbone of residue 8 in the compound of the invention.

According to a specific embodiment of the invention, if according to general formula I n8, n4, Y, and R⁸ together are —CH₂CH(CH₃)CH₃, R⁸¹ denotes CH₃, and n3 denotes 0, the amino acid (C_{α}-Me)Leu is formed by n8, n4, Y, and R⁸ together with the peptide backbone of residue 8.

According to a specific embodiment of the invention, if according to general formula I n8, n4, Y, and R⁸ together are —CH₂CH(CH₃)CH₃, R⁸¹ denotes H, and n3 denotes 1, the amino acid (β³-homo)Leu is formed by n8, n4, Y, and R⁸ together with the peptide backbone of residue 8.

According to the invention, non-limiting examples of residue 8 formed by R⁸, n4, n8, and Y together with the peptide backbone of residue 8 are Lys(Ac), diaminopropionic acid(Ac) (Dpr(Ac)), (Cα-Me)Leu, (β³-homo)Leu, Dpr(pivaloyl) (Dpr(Piv)).

The term “PEGylation” refers to the attachment of polyethylene glycol (PEG) chains to molecules. According to the invention, the degree of PEGylation is in the range of PEG₂₋₅₀. More specifically, the degree of PEGylation is in the range of PEG₂₋₅₀, PEG₁₀₋₅₀, PEG₁₅₋₅₀, PEG₂₀₋₅₀, PEG₂₅₋₅₀, PEG₃₀₋₅₀, PEG₃₅₋₅₀, PEG₄₀₋₅₀, PEG₄₅₋₅₀, PEG₂₋₅, PEG₂₋₁₀, PEG₂₋₁₅, PEG₂₋₂₀, PEG₂₋₂₅, PEG₂₋₃₀, PEG₂₋₃₅, PEG₂₋₄₀, PEG₂₋₄₅ PEG₁₀₋₂₀, PEG₂₀₋₃₀, or PEG₃₀₋₄₀.

The term “lipidation” refers to the attachment of a lipid group to a molecule. According to the invention, the lipid group can be a fatty acid or a fatty diacid. A fatty acid is a carboxylic acid with an aliphatic chain, which is either saturated or unsaturated. A fatty diacid is an α/ω carboxylic diacid with an aliphatic chain, which is either saturated or unsaturated, that connects the two carboxylic acid moieties at the α and ω termini. In the compound of the invention, the fatty acid or fatty diacid comprises aliphatic chains of 6 to 30 carbon atoms. More specifically, the fatty acid or the fatty diacid comprise aliphatic chains of 6, 7, 8, 9, 10, 11, 12, 13, 14, 15, 16, 17, 18, 19, 20, 21, 22, 23, 24, 25, 26, 27, 28, 29, or 30 carbon atoms.

According to another embodiment of the invention, terminal lipidation and PEGylation can be combined in that the compound of the invention has both lipidation and PEGylation. The PEG and lipid group can be interconnected by suitable amino acids, for example by amino acids Lys or Glu, optionally in the form of the β-amino acids, the γ-amino acids, the enantiomers, and the salts thereof.

The term “labelling” refers to the attachment of a chemical label to a molecule.

According to one embodiment of the invention, non-limiting examples of such chemical labels used for the labelling according to the invention are selected from the group consisting of biotin; fluorophores; antigens; metal chelators; MS-, NMR-, PET- and MRI-active tags; technical recognition tags; and biological recognition tags. Non-limiting examples of metal chelators are DOTA, NODA, and NODAGA. DOTA refers to 2,2′,2″,2′″-(1,4,7,10-tetraazacyclododecane-1,4,7,10-tetrayl)tetraacetic acid. NODA refers to 2,2′,2″-(1,4,7-triazacyclononane-1,4,7-triyl)triacetic acid). NODAGA refers to 1,4,7-triazacyclononane, 1-glutaric acid-4,7-acetic acid.

The term “terminal” in relation to PEGylation, lipidation, and labelling refers to the presence of the respective chemical moiety at the end of the side chain of residue 8. In other words, said terminal moiety is attached at the end of R⁸.

According to one embodiment of the invention, the chemical composition of residue 9 is defined in general formula I. Variable R⁹ defines possible modifications of the side chain of the amino acid of residue 9. Variables X³, R^{y}, and X⁴ define possible modifications of the peptide backbone of residue 9.

According to another embodiment of the invention, the C-terminus of the compound of the invention is formed by variable Z⁹ if m denotes 1. In the case m denotes 0, the C-terminus is formed by the C-terminal end of residue 8 comprising variables X³ and R^{y}.

According to a specific embodiment of the invention, the peptide backbone extension in the compound of the invention is defined herein by variables n1, n2, and n3. For example, peptide backbone extension refers to the formation of β-homo amino acids in the case n1, n2, and/or n3 denotes 1, to the formation of γ-homo amino acids in the case n1, n2, and/or n3 denotes 2, or to the formation of δ-homo amino acids in the case n1, n2, and/or n3 denotes 3.

According to a specific embodiment of the invention, a carbonyl replacement in the peptide backbone is defined herein by variable W.

According to a specific embodiment of the invention, Cα substitution in the peptide backbone is defined herein by variables R²¹, R³¹, R⁷¹, and R⁸¹. As used herein, if R²¹, R³¹, R⁷¹, or R⁸¹ denotes CH₃ at the respective residue, it is referred to as “Cα-Me”, e.g., (Cα-Me)Leu, (Cα-Me)Phe, (Cα-Me)Val, or (Cα-Me)Tyr.

According to a specific embodiment of the invention, Cα replacement in the peptide backbone is defined herein by variable X⁴.

According to a specific embodiment of the invention, N replacement in the peptide backbone is defined herein by variables Z¹, Z⁹, and X³.

According to a specific embodiment of the invention, N alkylation in the peptide backbone is defined herein by variables R^{x} and R^{y}.

In one embodiment of the invention, the compound of the general formula I is preferably the compound of the following formula II

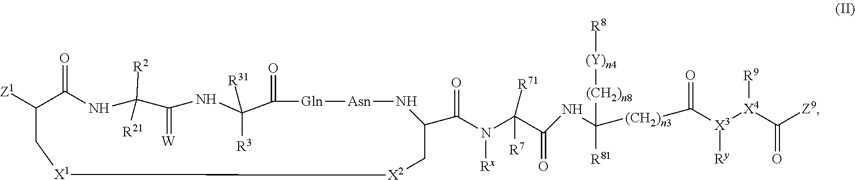

wherein Z¹, X¹, R², R²¹, W, R³, R³¹, X², R^{x}, R⁷, R⁷¹, R⁸, Y, n4, n8, R⁸¹, n3, R^{y}, X⁴, R⁹, and Z⁹ have the meanings given in the description and claims.

In one embodiment of the invention, the compound of general formula I is preferably the compound of the following formula III

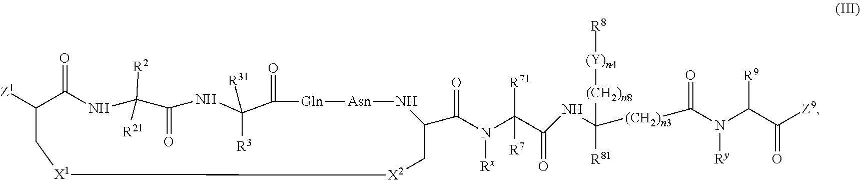

wherein Z¹, X¹, R², R²¹, R³, R³¹, X², R^{x}, R⁷, R⁷¹, R⁸, Y, n4, n8, R⁸¹, n3, R^{y}, R⁹, and Z⁹ have the meanings given in the description and claims.

In one embodiment of the invention, the compound of general formula I is preferably the compound of the following formula IV

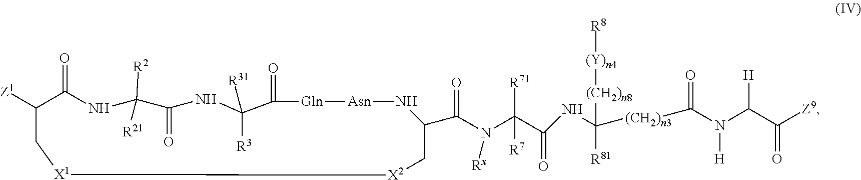

wherein Z¹, X¹, R², R²¹, R³, R³¹, X², R^{x}, R⁷, R⁷¹, R⁸, Y, n4, n8, R⁸¹, n3, and Z⁹ have the meanings given in the description and claims.

In one embodiment of the invention, the gut stability of the compound of the invention is different than the gut stability of OT.

According to one embodiment of the invention, in the compound of general formula I, if R⁸, n4, n8, and Y together with the peptide backbone of residue 8 is Ala or Thr, then R² together with the peptide backbone of residue 2 is different from Tyr, and/or R³ together with the peptide backbone of residue 3 is different from Ile, and/or one of X¹ or X² is different from S.

According to one embodiment of the invention, in the compound of general formula I, if R⁸, n4, n8, and Y together with the peptide backbone of residue 8 is Thr, then R² together with the peptide backbone of residue 2 is different from Tyr, and/or R³ together with the peptide backbone of residue 3 is different from Ile, and/or one of X¹ or X² is different from S.

According to one embodiment of the invention, in the compound of general formula I, the variables R⁸, n4, n8, and Y together with the peptide backbone of residue 8 do not form Thr.

According to one embodiment of the invention, in the compound of general formula I, if R⁸, n4, n8, and Y together with the peptide backbone of residue 8 is Ala, then R² together with the peptide backbone of residue 2 is different from Tyr, and/or R³ together with the peptide backbone of residue 3 is different from Ile, and/or one of X¹ or X² is different from S.

According to one embodiment of the invention, in the compound of general formula I, the variables R⁸, n4, n8, and Y together with the peptide backbone of residue 8 do not form Ala.

According to one embodiment of the invention, in the compound of general formula I, the variables R⁸, n4, n8, and Y together with the peptide backbone of residue 8 cannot form Pro. In other words, in the compound of general formula I, position 8 cannot be Pro.

According to one embodiment of the invention, in the compound of general formula I, the variables R⁸, n4, n8, and Y together with the peptide backbone of residue 8 do not form Cys, Cys conjugated to acetamide (Cys-AC), and/or Cys-CC(O)NH₂.

According to a specific embodiment of the invention, in the compound of general formula I, the variables R⁸, n4, n8, and Y together with the peptide backbone of residue 8 do not form Arg, Arg-NH₂, Arg-OH, Orn, Dab, Dap, and/or Cit.

According to one embodiment of the invention, in the compound of general formula I, if position 8 is any one of Arg, Arg-NH₂, Arg-OH, Orn, Dab, Dap, or Cit, then R² together with the peptide backbone of residue 2 is different from Tyr, and/or R³ together with the peptide backbone of residue 3 is different from Ile, and/or one of X¹ or X² is different from S.

According to one embodiment of the invention, in the compound of general formula I, position 8 is different from Arg, Arg-NH₂, Arg-OH, Orn, Dab, Dap, and/or Cit.

According to one embodiment of the invention, in the compound of general formula I, position 8 is different from Leu, lie, cyclopentyl-Gly (Cpg), Lys, Arg, and/or the enantiomers thereof.

According to one embodiment of the invention, in the compound of general formula I, R⁸, n4, n8, and Y together with the peptide backbone is different from Leu, lie, cyclopentyl-Gly (Cpg), Lys, Arg, and/or the enantiomers thereof.

According to a specific embodiment of the invention, in the compound of general formula I, R² together with the peptide backbone of residue 2 is different from Phe.

According to one embodiment of the invention, in the compound of general formula I, R² together with the peptide backbone of residue 2 is different from Trp, specifically from D-Trp.

According to a specific embodiment of the invention, in the compound of general formula I, X¹ or X² is different from S.

According to one embodiment of the invention, in the compound of general formula I, Z¹ denotes NH₂ or H.

According to one embodiment of the invention, in the compound of general formula I, Z⁹ denotes NH₂.

According to one embodiment of the invention, in the compound of general formula I, X² denotes S and X¹ denotes S, Se, or CH₂.

According to one embodiment of the invention, in the compound of general formula I, X¹ denotes S and X² denotes S, Se, or CH₂.

In one embodiment of the invention, the compound of general formula I is preferably the compound of the following formula V

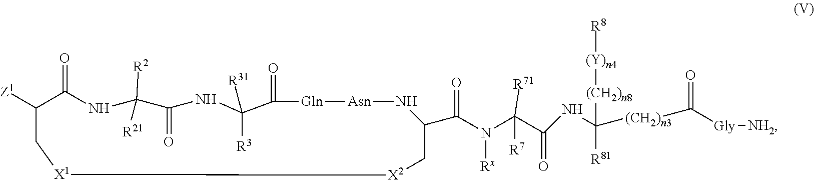

wherein Z¹, X¹, R², R²¹, R³, R³¹, X², R^{x}, R⁷, R⁷¹, R⁸, Y, n4, n8, R⁸¹, and n3 have the meanings given in the description and claims.

According to a specific embodiment of the invention, the compound of general formula I is the compound of the following formula VI,

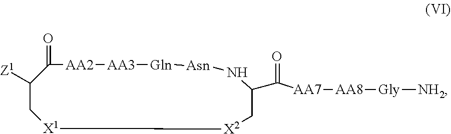

wherein
Z¹ denotes NH₂ or H;
X¹ and X² independently from one another denote S or CH₂;
AA2 denotes Tyr, Tyr(Me), (C_{α}-Me)Tyr, (C_{α}-Me)Phe, Phe(4-Me), Phe(4-CF₃), or Phe(4-F);
AA3 denotes lie, Cpg, (C_{α}-Me)Val, or Chg;
AA7 denotes Pro, (4-FBzl)Gly, thioPro, or Hyp;
AA8 denotes Lys(Ac), Dpr(Ac), (C_{α}-Me)Leu, (β³-homo)Leu, Dpr(Piv), or Lys(γ Glu, α Palm).

Specifically, the compound of the invention has an increased gut stability compared to OT.

According to one embodiment of the invention, the gut stability of the compound of the invention is increased by at least 10-fold, by 10-100-fold or by >100-fold compared to the gut stability of OT.

In another embodiment of the invention, the compound of the invention is used as a medicament.

According to a specific embodiment, the compound of the invention is used in the treatment or prevention of gastrointestinal disorders and/or abdominal/visceral pain.

According to a specific embodiment, the compound of the invention enables and is used in gut-specific treatment. A non-limiting example of a gut-specific treatment is the oral administration of the compound of the invention.

According to a specific embodiment, a gastrointestinal disorder as used herein refers to symptoms and/or causes associated with IBD including ulcerative colitis and Crohn's disease, and to symptoms and/or causes associated with IBS, including but not limited to abdominal pain, constipation, diarrhea, mixed, abnormal bowel movement, inflammation, weight loss, fatigue, and rectal bleeding.

### Definitions

As used herein, the following definitions apply, unless stated otherwise:

Unless specified otherwise, the term “alkyl”, when used alone or in combination with other groups or atoms, refers to a saturated straight or branched chain consisting solely of 1 to 6 hydrogen-substituted carbon atoms, and includes methyl, ethyl, propyl, isopropyl, n-butyl, 1-methylpropyl, isobutyl, t-butyl, 2,2-dimethylbutyl, 2,2-dimethyl-propyl, n-pentyl, 2-methylpentyl, 3-methylpentyl, 4-methylpentyl, n-hexyl and the like.

Unless specified otherwise, the term “alkenyl” refers to a partially unsaturated straight or branched chain consisting solely of 2 to 6 hydrogen-substituted carbon atoms that contains at least one double bond, and includes vinyl, allyl, 2-methylprop-1-enyl, but-1-enyl, but-2-enyl, but-3-enyl, buta-1,3-dienyl, penta-1,3-dienyl, penta-2,4-dienyl, 2-methylbut-1-enyl, 2-methylpent-1-enyl, 4-methylpent-1-enyl, 4-methylpent-2-enyl, 2-methylpent-2-enyl, 4-methylpenta-1,3-dienyl, hexen-1-yl and the like.

Unless specified otherwise, the term “alkynyl” refers to a partially unsaturated straight or branched chain consisting solely of 2 to 6 hydrogen-substituted carbon atoms that contains at least one triple bond, and includes ethynyl, 1-propynyl, 2-propynyl, 2-methylprop-1-ynyl, 1-butynyl, 2-butynyl, 3-butynyl, 1,3-butadiynyl, 3-methylbut-1-ynyl, 4-methylbut-ynyl, 4-methylbut-2-ynyl, 2-methylbut-1-ynyl, 1-pentynyl, 2-pentynyl, 3-pentynyl, 4-pentynyl, 1,3-pentadiynyl, 1,4-pentadiynyl, 3-methylpent-1-ynyl, 4-methylpent-2-ynyl, 4-methylpent-2-ynyl, 1-hexynyl, and the like.

Unless specified otherwise, the term “cycloalkyl”, when used alone or in combination with other groups or atoms, refers to monocyclic hydrocarbon rings, bicyclic hydrocarbon rings or spirohydrocarbon rings, which each may be either saturated or unsaturated (cycloalkenyl). The term unsaturated means that in the ring system in question there is at least one double bond, but no aromatic system is formed. In bicyclic hydrocarbon rings two rings are linked such that they have at least two carbon atoms in common. In spirohydrocarbon rings one carbon atom (spiroatom) is shared by two rings. If a cycloalkyl is substituted, the substitution may be mono- or polysubstitution in each case, at all the hydrogen-carrying carbon atoms, independently of one another. Cycloalkyl itself may be linked to the molecule as substituent via any suitable position of the ring system.

Typical examples of individual sub-groups are listed below. Monocyclic saturated hydrocarbon rings: cyclopropyl; cyclobutyl; cyclopentyl; cyclohexyl; cycloheptyl, etc.

Monocyclic unsaturated hydrocarbon rings: cycloprop-1-enyl; cycloprop-2-enyl; cyclobut-1-enyl; cyclobut-2-enyl; cyclopent-1-enyl; cyclopent-2-enyl; cyclopent-3-enyl; cyclohex-1-enyl; cyclohex-2-enyl; cyclohex-3-enyl; cyclohept-1-enyl; cyclohept-2-enyl; cyclohept-3-enyl; cyclohept-4-enyl; cyclobuta-1,3-dienyl; cyclopenta-1,4-dienyl; cyclopenta-1,3-dienyl; cyclopenta-2,4-dienyl; cyclohexa-1,3-dienyl; cyclohexa-1,5-dienyl; cyclohexa-2,4-dienyl; cyclohexa-1,4-dienyl; cyclohexa-2,5-dienyl, etc.

Saturated and unsaturated bicyclic hydrocarbon rings: bicyclo[2.2.0]hexyl; bicyclo[3.2.0]heptyl; bicyclo[3.2.1]octyl; bicyclo[2.2.2]octyl; bicyclo[4.3.0]nonyl (octahydroindenyl); bicyclo[4.4.0]decyl (decahydronaphthalene); bicyclo[2,2,1]heptyl (norbornyl); (bicyclo[2.2.1]hepta-2,5-dienyl (norborna-2,5-dienyl); bicyclo[2,2,1]hept-2-enyl (norbornenyl); bicyclo[4.1.0]heptyl (norcaranyl); bicyclo-[3.1.1]heptyl (pinanyl), etc.

Saturated and unsaturated spirohydrocarbon rings: spiro[2.5]octyl, spiro[3.3]heptyl, spiro[4.5]dec-2-ene, etc.

“Cycloalkylalkyl” denotes the combination of the above-defined groups alkyl, alkenyl, alkynyl, and cycloalkyl, in each case in their broadest sense. The alkyl group as substituent is directly linked to the molecule and is in turn substituted by a cycloalkyl group. The alkyl and cycloalkyl may be linked in both groups via any carbon atoms suitable for this purpose. The respective sub-groups of alkyl and cycloalkyl are also included in the combination of the two groups.

Unless specified otherwise, the term “aryl” refers to an aromatic mono- or bicyclic group containing from 6 to 14 carbon atoms, preferably 6 to 10 carbon atoms, that may be optionally fused with a fully or partially saturated or unsaturated carbocyclic ring and may optionally be substituted with one or more identical or different substituents, suitably one to three substituents. Examples of aryl groups include phenyl, naphthyl, indanyl, and the like.

“Arylalkyl” denotes the combination of the groups alkyl, alkenyl, alkynyl and aryl as hereinbefore defined, in each case in their broadest sense. The alkyl group as substituent is directly linked to the molecule and is in turn substituted by an aryl group. The alkyl and aryl may be linked in both groups via any carbon atoms suitable for this purpose. Typical examples include benzyl, 1-phenylethyl, 2-phenylethyl, phenylvinyl, phenylallyl, etc.

Unless specified otherwise, the term “heteroaryl” refers to an aromatic mono- or bicyclic group containing from 5 to 14 carbon atoms, preferably 5 to 12 carbon atoms, of which one to five is replaced with a heteroatom selected from N, S and O, that may optionally be reduced to a non-aromatic heterocycle and may optionally be substituted with one or more identical or different substituents. Examples of heteroaryl groups include pyrrolyl, dihydropyrrolyl, pyrrolidinyl, oxopyrrolidinyl, indolyl, isoindolyl, indolizinyl, imidazolyl, pyrazolyl, benzimidazolyl, imidazo(1,2-a)pyridinyl, indazolyl, purinyl, pyrrolo(2,3-c)pyridinyl, pyrrolo(3,2-c)pyridinyl, pyrrolo(2,3-b)pyridinyl, pyrazolo(1,5-a)pyridinyl, 1,2,3-triazolyl, 1,2,4-triazolyl, tetrazolyl, oxazolyl, 1,2 oxazolyl, isoxazolyl, 1,3,4-oxadiazolyl, 1,2,5-oxadiazolyl, 1,2,4-oxadiazolyl, 1,2,3-oxadiazolyl, thiazolyl, isothiazolyl, 1,3,4-thiadiazolyl, 1,2,5-thiadiazolyl, 1,2,4-thiadiazolyl, 1,2,3-thiadiazolyl, furanyl, dihydrofuranyl, tetrahydrofuranyl, benzofuranyl, isobenzofuranyl, thiophenyl, dihydrothiophenyl, tetrahydrothiophenyl, benzothiophenyl, benzoisothiophenyl, pyridyl, piperidinyl, quinolinyl, isoquinolinyl, tetrahydroisoqinolinyl, quinolizinyl, pyrazinyl, pyridazinyl, pyrimidinyl, pyranyl, tetrahydropyranyl, 1,2,3-triazinyl, 1,2,4-triazinyl, 1,3,5-triazinyl, chromenyl, morpholinyl, diazepinyl, benzodiazepinyl, and the like.

“Heteroarylalkyl” denotes the combination of the alkyl, alkenyl, alkynyl, and heteroaryl groups defined hereinbefore, in each case in their broadest sense. The alkyl group as substituent is directly linked to the molecule and is in turn substituted by a heteroaryl group. The linking of the alkyl and heteroaryl may be achieved on the alkyl side via any carbon atoms suitable for this purpose and on the heteroaryl side by any carbon or nitrogen atoms suitable for this purpose.

By the term “heterocycloalkyl” are meant groups which are derived from cycloalkyl as hereinbefore defined if in the hydrocarbon rings one or more of the groups —CH₂— are replaced independently of one another by the groups —O—, —S— or —NH— or one or more of the groups ═CH— are replaced by the group ═N—, while not more than five heteroatoms may be present in total, there must be at least one carbon atom between two oxygen atoms and between two sulfur atoms or between one oxygen and one sulfur atom and the group as a whole must be chemically stable. Heteroatoms may simultaneously be present in all the possible oxidation stages (sulfur->sulfoxide —SO—, sulfone —SO₂—; nitrogen->N-oxide). It is immediately apparent from the indirect definition/derivation from cycloalkyl that heterocycloalkyl is made up of the sub-groups monocyclic hetero-rings, bicyclic hetero-rings and spirohetero-rings, while each sub-group can also be further subdivided into saturated and unsaturated (heterocycloalkenyl). The term unsaturated means that in the ring system in question there is at least one double bond, but no aromatic system is formed. In bicyclic hetero-rings two rings are linked such that they have at least two atoms in common. In spirohetero-rings one carbon atom (spiroatom) is shared by two rings. If a heterocycloalkyl is substituted, the substitution may be mono- or polysubstitution in each case, at all the hydrogen-carrying carbon and/or nitrogen atoms, independently of one another. Heterocycloalkyl itself as substituent may be linked to the molecule via any suitable position of the ring system.

The term “heterocyclic group” as used herein refers to a heterocycloalkyl group which optionally may be fused to an aromatic aryl or heteroaryl group.

Typical examples of individual sub-groups are listed below: Monocyclic heterorings (saturated and unsaturated): oxolane, pyrrolidinyl, pyrrolinyl, imidazolidinyl, thiazolidinyl, imidazolinyl, pyrazolidinyl, pyrazolinyl, piperidinyl, piperazinyl, oxiranyl, aziridinyl, azetidinyl, 1,4-dioxanyl, azepanyl, diazepanyl, morpholinyl, thiomorpholinyl, homomorpholinyl, homopiperidinyl, homopiperazinyl, homothiomorpholinyl, thiomorpholinyl-S-oxide, thiomorpholinyl-S,S-dioxide, 1,3-dioxolanyl, oxane, tetrahydrothiopyranyl, 1,4-oxazepanyl, tetrahydrothienyl, homothiomorpholinyl-S,S-dioxide, oxazolidinonyl, dihydropyrazolyl, dihydropyrrolyl, dihydropyrazinyl, dihydropyridyl, dihydro-pyrimidinyl, dihydrofuryl, dihydropyranyl, tetrahydrothienyl-S-oxide, tetrahydrothienyl-S,S-dioxide, homothiomorpholinyl-S-oxide, 2,3-dihydroazet, 2H-pyrrolyl, 4H-pyranyl, 1,4-dihydropyridinyl, etc;

Bicyclic heterorings (saturated and unsaturated): 8-azabicyclo[3.2.1]octyl, 8-azabicyclo[5.1.0]octyl, 2-oxa-5-azabicyclo[2.2.1]heptyl, 8-oxa-3-aza-bicyclo[3.2.1]octyl, 3,8-diaza-bicyclo[3.2.1]octyl, 2,5-diaza-bicyclo-[2.2.1]heptyl, 1-aza-bicyclo[2.2.2]octyl, 3,8-diaza-bicyclo[3.2.1]octyl, 3,9-diaza-bicyclo[4.2.1]nonyl, 2,6-diaza-bicyclo[3.2.2]nonyl, hexahydro-furo[3,2-b]furyl, etc; Spiro-heterorings (saturated and unsaturated): 1,4-dioxa-spiro[4.5]decyl; 1-oxa-3,8-diaza-spiro[4.5]decyl; 2,6-diaza-spiro[3.3]heptyl; 2,7-diaza-spiro[4.4]nonyl; 2,6-diaza-spiro[3.4]octyl; 3,9-diaza-spiro[5.5]undecyl; 2,8-diaza-spiro[4.5]decyl, etc.

“Heterocycloalkylalkyl” denotes the combination of the alkyl, alkenyl, alkynyl, and heterocycloalkyl groups defined hereinbefore, in each case in their broadest sense. The alkyl group as substituent is directly linked to the molecule and is in turn substituted by a heterocycloalkyl group. The linking of the alkyl and heterocycloalkyl may be achieved on the alkyl side via any carbon atoms suitable for this purpose and on the heterocycloalkyl side by any carbon or nitrogen atoms suitable for this purpose.

By the term “suitable substituent” is meant a substituent that on the one hand is fitting on account of its valency and on the other hand leads to a system with chemical stability.

It is intended that the definition of any substituent or variable at a particular location in a molecule be independent of its definitions elsewhere in that molecule. It is understood that substituents and substitution patterns on the compounds of this invention can be selected by one of ordinary skill in the art to provide compounds that are chemically stable and that can be readily synthesized by techniques known in the art as well as those methods set forth herein.

The term “tautomers” refer to compounds that are interchangeable forms of a particular compound structure, and that vary in the displacement of hydrogen atoms and electrons. Thus, two structures may be in equilibrium through the movement of π electrons and an atom (usually H). For example, enols and ketones are tautomers because they are rapidly interconverted by treatment with either acid or base.

It is also to be understood that compounds (e.g., dihydro bases described herein) that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively).

A 50:50 mixture of enantiomers is referred to as a racemic mixture or a racemate, which may occur where there has been no stereoselection or stereospecificity in a chemical reaction or process. The terms “racemic mixture” and “racemate” refer to an equimolar mixture of two enantiomeric species, devoid of optical activity.

The term “hydrate” refers to a compound which is associated with water. Typically, the number of the water molecules contained in a hydrate of a compound is in a definite ratio to the number of the compound molecules in the hydrate.

Any formula or structure given herein, including Formula I compounds, is also intended to represent unlabeled forms as well as isotopically-labeled forms of the compounds. Isotopically-labeled compounds have structures depicted by the formulas given herein except that one or more atoms are replaced by an atom having a selected atomic mass or mass number. Examples of isotopes that can be incorporated into compounds of the invention include isotopes of hydrogen, carbon, nitrogen, oxygen, phosphorous, fluorine, and chlorine, such as, but not limited to ²H (deuterium, D), ³H (tritium), ¹¹C, ¹³C, ¹⁴C, ¹⁵N, ¹⁸F, ³¹P, ³¹P, ³⁵S, ³⁶Cl, and ¹²⁵I.

The term “pharmacologically acceptable” means compatible with the treatment of animals, in particular, humans. The term pharmacologically acceptable salt includes both pharmacologically acceptable acid addition salts and pharmacologically acceptable basic addition salts.

The term“pharmacologically acceptable acid addition salt” as used herein means any non-toxic organic or inorganic salt of any base compound of the disclosure, or any of its intermediates. Basic compounds of the disclosure that may form an acid addition salt include, for example, compounds that contain a basic nitrogen atom. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methane sulfonic acids. Either the mono-, di- or the triacid salts can be formed, and such salts may exist in either a hydrated, solvated or substantially anhydrous form. In general, the acid addition salts of the compounds of the disclosure are more soluble in water and various hydrophilic organic solvents, and generally demonstrate higher melting points in comparison to their free base forms. The selection of the appropriate salt will be known to one skilled in the art. Other non-pharmacologically acceptable acid addition salts, e.g. oxalates, may be used, for example, in the isolation of the compounds of the disclosure, for laboratory use, or for subsequent conversion to a pharmacologically acceptable acid addition salt.

The term “pharmacologically acceptable basic salt” as used herein means any non-toxic organic or inorganic basic addition salt of any acid compound of the invention, or any of its intermediates, which are suitable for or compatible with the treatment of animals, in particular humans. Acidic compounds of the invention that may form a basic addition salt include, for example compounds that contain carboxylic acid, sulfonic acid, sulfinic acid, sulfonamide, N-unsubstituted tetrazole, phosphoric acid ester, or sulfuric acid ester. Illustrative inorganic bases which form suitable salts include lithium, sodium, potassium, calcium, magnesium, or barium hydroxide. Illustrative organic bases which form suitable salts include aliphatic, alicyclic or aromatic organic amines such as methylamine, trimethylamine and picoline or ammonia. The selection of the appropriate salt will be known to a person skilled in the art. Other non-pharmacologically acceptable basic addition salts, may be used, for example, in the isolation of the compounds of the invention, for laboratory use, or for subsequent conversion to a pharmacologically acceptable basic addition salt. The formation of a desired compound salt is achieved using standard techniques. For example, the neutral compound is treated with a base in a suitable solvent and the formed salt is isolated by filtration, extraction or any other suitable method.

The term “therapeutically effective amount”, “effective amount” or “sufficient amount” of a compound of the present invention is a quantity sufficient to, when administered to the subject, including a mammal, for example a human, effect beneficial or desired results, including clinical results, and, as such, an effective amount or synonym thereof depends upon the context in which it is being applied.

The terms “treatment”, “treat” or “treating” or “therapy” refer to clinical intervention in an attempt to alter the natural course of the individual being treated, and treatment can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

As used herein and in the claims, the singular form, for example “a”, “an” and “the” includes the plural, unless the context clearly dictates otherwise.

The terms “comprise”, “contain”, “have” and “include” as used herein can be used synonymously and shall be understood as an open definition, allowing further members or parts or elements. “Consisting” is considered as a closest definition without further elements of the consisting definition feature. Thus “comprising” is broader and contains the “consisting” definition.

Unless indicated or defined otherwise, all terms used herein have their usual meaning in the art, which will be clear to the skilled person.

### EXAMPLES

The Examples which follow are set forth to aid in the understanding of the invention but are not intended to, and should not be construed to, limit the scope of the invention in any way. The Examples do not include detailed descriptions of conventional methods. Such methods are well known to those of ordinary skill in the art.

### General Experimental Details

### Materials

All reagents and solvents were commercially obtained in analytical or peptide synthesis grade and used without further purification. Standard protected 9-Fluorenylmethoxycarbonyl (Fmoc)-amino acids, Fmoc-S-p-methoxytrityl-L-cysteine (Fmoc-Cys(Mmt)-OH), Boc-S-trityl-L-cysteine (Boc-Cys(Trt)-OH), Fmoc-alpha-methyl-L-phenylalanine (Fmoc-(C_{α}-Me)Phe-OH), Fmoc-thiazolidine-4-carboxylic acid (Fmoc-thioPro-OH), N,N′-diisopropylcarbodiimide (DIC), Oxyma Pure and Fmoc-Rink amide AM resin (0.74 mmol/g, 100-200 mesh) were purchased from Iris Biotech GmbH (Marktredwitz, Germany). TentaGel R RAM (Rink amide) resin (0.19 mmol/g, particle size: 90 μM) was purchased from Rapp Polymere (Tübingen, Germany). 1-[Bis(dimethylamino)methylene]-1H-1,2,3-triazolo[4,5-b]pyridinium 3-oxide hexafluorophosphate (HATU), 4-bromobutyric acid, phenylsilane (PhSiH₃), Fmoc-L-Cyclohexylglycine (Fmoc-Chg-OH), Fmoc-L-Cyclopentylglycine (Fmoc-Cpg-OH), Fmoc-alpha-methyl-L-leucine (Fmoc-(C_{α}-Me)Leu-OH), Fmoc-O-methyl-L-tyrosine (Fmoc-Tyr(Me)-OH), Fmoc-4-fluor-L-phenylalanin (Fmoc-Phe(4-F)—OH), Fmoc-(4-trifluoromethyl)-L-phenylalanine (Fmoc-Phe(4-CF₃)—OH) and 4-fluorobenzylamine were purchased from Fluorochem Ldt. (Derbyshire, UK). Dichloromethane (DCM), N,N-dimethylformamide (DMF), diethyl ether (Et₂O), ethyl acetate (EtOAc), heptane and trifluoroacetic acid (TFA) were purchased from VWR International (Darmstadt, Germany). Piperidine, N,N-diisopropylethylamine (DIEA), triisopropylsilane (TIPS), 2,2′-(ethylendioxy)diethanethiol (EDT), dimethyl sulfide (DMS), 4-bromobutyryl chloride, Fmoc-N-ε-acetyl-L-lysine (Fmoc-Lys(Ac)-OH), Fmoc-N-β-4-methyltrityl-L-diaminopropionic acid (Fmoc-Dpr(Mtt)-OH), Fmoc-N-ε-Fmoc-L-lysine (Fmoc-Lys(Fmoc)-OH), Fmoc-L-trans-4-hydroxyprolin (Fmoc-Hyp(tBu)-OH), bromoacetic acid, trimethylacetic anhydride (pivalic anhydride and allyl alcohol were purchased from Sigma-Aldrich, Merck (Darmstadt, Germany). Fmoc-N-ε-4-methyltrityl-L-lysine (Fmoc-Lys(Mtt)-OH) was purchased from GL Biochem (Shanghai, China). Fmoc-4-methyl-L-phenylalanine (Fmoc-Phe(4-Me)-OH) and Fmoc-β³-homoleucine (Fmoc-(β³-homo)Leu-OH) were purchased from Alfa Aesar, Thermo Fisher Scientific (Kandel, Germany), Fmoc-alpha-methyl-O-t-butyl-L-tyrosine (Fmoc-(C_{α}-Me)Tyr(tBu)-OH) from Syntides, Psyclo Peptide (Shanghai, China) and Fmoc-alpha-methyl-L-valine (Fmoc-(C_{α}-Me)Val-OH) from Bachem (Bubendorf, Switzerland). (Benzotriazol-1-yloxy)tripyrrolidinophosphonium hexafluorophosphate (PyBOP) was purchased from Carbosynth Ltd, (Compton, Berkshire, UK). Acetonitrile (ACN) and formic acid were purchased from VWR International (Darmstadt, Germany). Pancreatin from porcine pancreas (1×USP) was purchased from VWR International (Darmstadt, Germany). Pepsin from porcine gastric mucosa (1200-2400 U/mg) was purchased from Sigma-Aldrich, Merck (Darmstadt, Germany). KH₂PO₄, NaCl, NaOH pellets and HCl (6 M) were purchased from VWR International (Darmstadt, Germany). Double-distilled Milli-Q water (ddH₂O) was used for all buffer preparations. Dulbecco's Modified Eagle's Medium (DMEM) and penicillin/streptomycin were purchased from Thermo Fisher Scientific (Scoresby, Australia). Foetal Bovine Serum (FBS) was purchased from GE Life Sciences (Parramatta, Australia). The IP-1 assay kit was purchased from CisBio (Codolet, France) and HEK293 and COS-1 cells from American Type Culture Collection (ATCC, Manassas, VA, USA).

### Example 1—Synthesis of Compounds

### General Peptide Synthesis and Purification

Peptides were manually assembled on a 0.05 mmol scale employing Fmoc-SPPS protocols as previously described [4]. In brief, standard amino acid couplings were performed with 5 eq. excess of amino acid, 5 eq. HATU (0.5 M) and 6 eq. DIPEA in DMF as a solvent (coupling time: 10 min). Fmoc was deprotected with 50% piperidine in DMF (1 min, twice). Unless otherwise stated, standard orthogonal protected Fmoc-amino acids were used as follows: Fmoc-Asn(Trt)-OH, Fmoc-Cys(Trt)-OH, Fmoc-Gln(Trt)-OH, Fmoc-Tyr(tBu)-OH. Upon assembly of the target sequences, dried peptide resins were treated with TFA:TIPS:EDT:DMS=94:2:2:2 to deprotect side chains and cleave the peptides from the solid support (cleavage time: 90 min). TFA was removed under continuous nitrogen stream and ice-cold Et₂O was added to precipitate the compounds. Crude peptides were washed twice with fresh ice-cold Et₂O (resuspended and centrifuged), dissolved in 1:1 ddH₂O/ACN containing 0.1% TFA and freeze dried. Linear sequences were cyclized using either cyclization method A, method B or method C. Specific synthesis and cyclization procedures are detailed individually for each derivative class. Peptides were purified via preparative RP-HPLC on a Waters Auto Purification HPLC-UV system equipped with a Kromasil Classic C₄ or C₁₈ column (21.2×250 mm, 300 Å, 10 μm) and UV detection at 214 nm. The following chromatographic parameters were used: flowrate of 20 mL/min and linear gradient elution of 5-55% solvent B in 50 min. Solvent A: 0.1% TFA in ddH₂O, solvent B: 0.08% TFA in ACN. All synthesized final compounds were purified to >95% as determined by analytical RP-HPLC and relative peak quantification at 214 nm.

### Peptide Analysis and Quality Control Via HPLC and (HR)-ESI-MS Analysis

Analytical HPLC chromatograms were recorded on a Thermo Scientific Vanquish Horizon UHPLC system with UV detection at 214 and 280 nm. The analysis was performed on a Kromasil Classic C₁₈ column (4.6×150 mm, 300 Å, 5 μm) using the following chromatographic parameters: linear gradient elution (5-65% solvent B in 30 min) and a flow rate of 1 mL/min at 30° C. Solvent A: 0.1% TFA in ddH₂O, solvent B: 0.08% TFA in ACN. High-resolution (HR)-MS analysis was performed on a Thermo Scientific LTQ Orbitrap Velos mass spectrometer coupled to a Thermo Scientific Vanquish Horizon UHPLC system. Samples were analyzed in LC-MS mode using an Acclaim C₁₈ HPLC column (2.1×150 mm, 120 Å, 3 μm, Thermo Fisher Scientific) and the following chromatographic parameters: linear gradient elution (10-65% solvent B in 14 min) and a flow rate of 0.45 mL/min at 30° C. Solvent A: 0.1% formic acid in ddH₂O, solvent B: 0.1% formic acid in ACN. HR-ESI-MS spectra were recorded in positive ion mode in the range of m/z 300-2000 with an FT resolution of 60,000. The sum formulas of the detected ions were confirmed using Xcalibur 4.2.47 based on the mass accuracy (Δm/z≤5 ppm) and isotopic pattern.

### Cyclization Method A

Linear sequences were manually assembled on an Fmoc-Rink amide AM resin (0.74 mm/g, swelled in DMF for 2 h) using the following cysteine side chain protecting groups: Cys¹(Trt) and Cys⁶(Trt). Linear peptides were cleaved from the solid support and isolated using standard conditions described under general peptide synthesis and purification. Crude linear precursor peptides were dissolved in aq. 0.1 M NH₄HCO₃ at pH 8.2 (peptide concentration: 200 μM) and stirred at air (25° C.) until complete cyclization was indicated by analytical HPLC-MS analysis. Folded products were isolated via preparative RP-HPLC using the general purification conditions described above.

### Cyclization Method B

Linear sequences were manually assembled on a TentaGel R RAM (Rink amide) resin (0.19 mmol/g, swelled in DCM for 1 h and in DMF for 1 h). Upon incorporation of Fmoc-Cys⁶(Mmt)-OH, the peptide-resin was treated with 1% TFA and 3% TIPS in DCM (5 min, 8 times). The resin was extensively washed with DMF and treated with allyl-4-bromobutanoate (10 eq., 1 M in DMF) and DIPEA (15 eq.) in a closed reaction vessel under argon atmosphere (1 h). The resin was extensively washed with DMF, and the remaining sequence was manually assembled using standard coupling conditions. Upon coupling of the last amino acid (position 2), the peptide resin was treated with Pd(PPh₃)₄ (1 eq., 0.05 M) and phenylsilane (50 eq.) in dry DCM in a closed reaction vessel under argon atmosphere (15 min, twice). The resin was extensively washed with DCM and DMF and Fmoc was removed with 50% piperidine in DMF (1 min, twice). The resin was extensively washed with DMF. PyBOP (2.5 eq., 0.5 M in DMF) and DIPEA (5 eq.) were added and incubated with the resin for 1 h. Final products were cleaved from the solid support, isolated and purified using standard conditions described under general peptide synthesis and purification.

### Cyclization Method C

Linear sequences were manually assembled on a TentaGel R RAM (Rink amide) resin (0.19 mmol/g, swelled in DCM for 1 h and in DMF for 1 h). Cysteine was incorporated as Fmoc-Cys⁶(Mmt)-OH and 4-bromobutyric acid (10 eq.) was coupled as the last residue using DIC (10 eq.)/Oxyma Pure (10 eq., 0.5 M in DMF) mediated activation (1 h, twice). Upon assembly of the sequence, the resin was treated with 1% TFA and 3% TIPS in DCM (5 min, 8 times). The resin was extensively washed with DCM and DMF and treated with DIPEA (10 eq.) in DMF (0.5 M) in a closed reaction vessel under argon atmosphere (overnight). Final products were cleaved from the solid support, isolated and purified using standard conditions described under general peptide synthesis and purification.

### Synthesis of Allyl-4-Bromobutanoat

In a Schlenk flask under argon atmosphere, a solution of allyl alcohol (1 eq.) and DIPEA (1.1 eq.) in dry chloroform (1 M) was stirred under ice-bath cooling. 4-bromobutyryl-chloride (1.1 eq.) was added dropwise over 15 min. The ice bath was removed and the mixture was let to room temperature and stirred for 4 h (reaction control via TLC, EtOAc:heptane=1:4). The solution was diluted with DCM and washed with aq. HCl (0.5 M, 3 times), aq. NaOH (0.5 M, 3 times) and brine, dried over MgSO₄ and the solvent was removed in vacuum. The crude product was obtained as a yellowish oil which was used without further purification [5].

### Synthesis of OT, 1, 3 and 4

Compounds were accessed using cyclization method A and general peptide synthesis and purification procedures. The following commercial non-standard amino acid building blocks were used: Fmoc-Lys(Ac)-OH, Fmoc-(C_{α}-Me)Leu-OH and Fmoc-(β³-homo)Leu-OH.

### Synthesis of 2

The compound was accessed using cyclization method A. Upon coupling of Fmoc-Dpr⁸(Mtt)-OH, the peptide-resin was treated with 1% TFA and 3% TIPS in DCM (5 min, 8 times) and the resin was extensively washed with DMF. The Dpr⁸ β-amine side chain was acetylated by treating the peptide-resin with acetic anhydride (35 eq., 0.6 M) and DIPEA (55 eq) in DMF (15 min). The remaining sequence was synthesized using standard conditions described under general peptide synthesis and purification and conditions specified for cyclization method A.

### Synthesis of 5, 7, 16, 19 and 21

Compounds were accessed using cyclization method B and general peptide synthesis and purification procedures. The following commercial non-standard amino acid building blocks were used: Fmoc-Lys(Ac)-OH, Fmoc-(C_{α}-Me)Leu-OH, Fmoc-Tyr(Me)-OH, Fmoc-Cpg-OH, Fmoc-(C_{α}-Me)Tyr-OH and Fmoc-(C_{α}-Me)Val-OH.

### Synthesis of 6, 9, 10, 11, 12, 13, 14, and 15

Compounds were accessed using cyclization method B. Upon coupling of Fmoc-Dpr⁸(Mtt)-OH, the peptide-resin was treated with 1% TFA and 3% TIPS in DCM (5 min, 8 times) and the resin was extensively washed with DMF. The unprotected Dpr⁸ β-amine side chain was acetylated by treating the peptide-resin with acetic anhydride (35 eq., 0.6 M) (pivalic anhydride was used for compound 13) and DIPEA (55 eq.) in DMF (15 min). The remaining sequence was synthesized using standard conditions described under general peptide synthesis and purification and conditions specified for cyclization method B.

### Synthesis of 8

The compound was accessed using cyclization method B. Upon coupling of Fmoc-Dpr⁸(Mtt)-OH, the peptide-resin was treated with 1% TFA and 3% TIPS in DCM (5 min, 8 times) and the resin was extensively washed with DMF. The unprotected Dpr⁸ β-amine side chain was acetylated by treating the peptide-resin with acetic anhydride (35 eq., 0.6 M) and DIPEA (55 eq) in DMF (15 min). The resin was extensively washed with DMF and bromoacetic acid (10 eq.) was incorporated using DIC (10 eq.)/Oxyma Pure (10 eq., 0.5 M in DMF) mediated activation (1 h). 4-fluorobenzylamin (10 eq., 0.5 M) in DMF was added and incubated with the resin for 1 h [6-7]. The remaining sequence was synthesized using standard conditions described under general peptide synthesis and purification and conditions specified for cyclization method B.

### Synthesis of 17, 18, 20, 22, 23, 24, and 25.

Compounds were accessed using cyclization method C and general peptide synthesis and purification procedures. The following commercial non-standard amino acid building blocks were used: Fmoc-Lys(Ac)-OH, Fmoc-thioPro-OH, Fmoc-Tyr(Me)-OH, Fmoc-Cpg-OH, Fmoc-(C_{α}-Me)Tyr-OH, Fmoc-(C_{α}-Me)Val-OH, Fmoc-Chg-OH, Fmoc-Hyp(tBu)-OH and Fmoc-Phe(4-F)—OH.

### Synthesis of 26

Compound 26 is accessed using cyclization method C and general peptide synthesis and purification procedures. The following commercial non-standard building blocks are used: Fmoc-thioPro-OH, Fmoc-(C_{α}-Me)Tyr-OH, Fmoc-Glu-OtBu, Fmoc-Lys(ivDde)-OH and palmitic acid. Lys is incorporated in position 8 as Fmoc-Lys⁸(ivDde)-OH. Upon complete assembly and cyclization, the peptide-resin is treated with 3% hydrazine in DMF and the resin is extensively washed with DMF. Fmoc-Glu-OtBu is coupled using HATU and DIPEA in DMF and Fmoc is deprotected with 50% piperidine in DMF. Palmitic acid is coupled using HATU and DIPEA in DMF and the final product is cleaved from the solid support, isolated and purified using standard conditions described under general peptide synthesis and purification. Position 8 in compound 26 features a Lys8 side chain that is modified with Glu and palmitic acid. The structure of position 8 in the peptide according to compound 26 is the following:

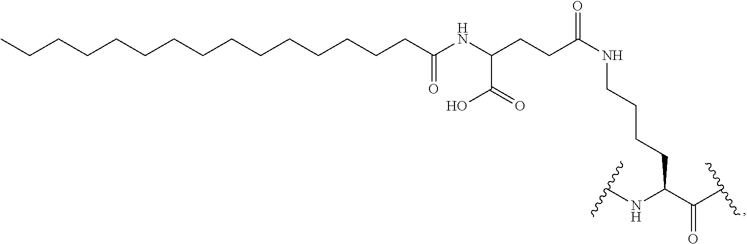 [SMILES: *N[C@@H](CCCCNC(=O)CCC(NC(=O)CCCCCCCCCCCCCCC)C(=O)O)C(*)=O]

### Example 2—Stability of Compounds

Simulated intestinal fluid (SIF). SIF composition met test solution criteria specified by the USP (USP 42—NF 37, 2019). Preparation of 10 mL fluid, pH 6.8: KH₂PO₄ (68 mg, 6.8 mg/mL) was dissolved in ddH₂O and the final pH (±0.1) was adjusted with aq. 3 M NaOH to give 10 mL solution at pH 6.8. Pancreatin (100 mg, 1×USP) was added, and the mixture was vortexed for 1 min and sonicated for 15 min at 25° C. The solution was centrifuged, and syringe filtered before use.

Stability assay procedure. Stock solutions (1 mM) of test peptides were prepared in ddH₂O. SIF (570 μL) was pre-incubated in a thermo shaker at 37° C. for 15 min. The peptide stock solution (30 μL) was added to the fluid, the mixture was vortexed and incubated at 37° C. Samples (30 μL) were drawn at time points 0, 2.5, 5, 15, 30, 60 min for all compounds and additionally at 2, 4, 6, 24 h for compounds with t_{1/2}>60 min and quenched by adding to ice-cold stop solution (30 μL, 5 vol % aq. TFA). All samples were centrifuged (5 min, 16,000×g) and stored at 4° C. before analysis.

RP-HPLC-UV(-MS) analysis of stability samples. Analysis was performed on a Dionex Ultimate 3000 system equipped with a UV-VIS detector (214 nm and 280 nm). 30 μL sample were injected on a Kromasil Classic C₁S HPLC column (2.1×150 mm, 100 Å, 5 μm) equipped with a guard column (C₁₈, 100 Å, 5 μm, 2.1 mm). Gradient elution (5-65% solvent B in 6 min, 10% B/min) and a flow rate of 1 mL/min at 30° C. were used. Solvent A: 0.1% TFA in ddH₂O. Solvent B: 0.08% TFA in ACN.

Data analysis. Data was analyzed by manual peak integration at 214 nm. Peak areas (mAU×min) at individual time points were normalized to the mean value of time point zero (y(t₀)=100%). To calculate compound half-lives (t_{1/2}), a one-phase exponential decay function was fitted to normalized data points via a nonlinear regression in GraphPad Prism (Version 9) (GraphPad Software Inc., La Jolla, California, USA). The following constraints were applied: (i) y(t₀) constant equal to 100, (ii) plateau constant equal to 0.

### Example 3—Biological Activity of Compounds

Cell culture for functional assays. Human OTR cDNA sequence was inserted into the pEGFP-N1 plasmid (Clontech, Mountain View, California, USA) to yield a hOTR-GFP fusion protein. A stably transfected HEK293 cell line was established using the jetPrime transfection protocol (Polyplus transfection) and G418 selection as per the manufacturers' protocols. Cells stably expressing the receptors were used for functional assays [8].

Measurement of inositol phosphate one (IP-1) accumulation. HEK293 cells were cultured in DMEM supplemented with 10% FBS and penicillin/streptomycin (100 U mL⁻¹) at 37° C. and 5% CO₂. Cells were transferred into white opaque 384-well plates (CellStar®, Greiner, Austria) at ˜10,000 cells per well 48 h prior to measurement. Quantitative measurements of receptor-mediated IP-1 were performed by competitive immunoassay utilizing the IP-One assay kit [9]. HEK293 cells were assayed as per the manufacturer's protocol. Briefly, at the time of analysis, the culture media was removed and replaced with IP-1 stimulation buffer. Cells were allowed to equilibrate in the stimulation buffer at 37° C. for 15 min, followed by addition of peptide ligands (to final concentrations of 1 μM-10 μM) and subsequent stimulation for 1 h at 37° C. The stimulation was terminated by lysis and the simultaneous addition of homogenous time-resolved fluorescence resonance energy transfer reagents. The lysates were incubated for 1 h at 25° C. Fluorescence emission measurements at 620 nm and 665 nm were performed using a Spark Multimode plate reader (Tecan, Switzerland) at an excitation wavelength of 340 nm. Results were analyzed as a ratio of fluorescence intensities of 665 nm to 620 nm.

Statistics and data analysis. IP-1 accumulation data was normalized to maximum OT response. Data was fitted via non-linear regression in GraphPad Prism (GraphPad Software Inc., La Jolla, California, USA) using a three-parameter modified Hill equation with a slope set to unity: Y=Bottom+(Top−Bottom)/(1+10^{LogEC50-X}), where X corresponds to the log of concentration; values for Top, Bottom and LogEC₅₀ were not constrained.

Described OT derivatives revealed high gut stability and potent biological activity at OTR (Table 1).

Table 1 Structures, gut stability and biological activity of example compounds. The SIF stability of the compounds is shown as relative stability in comparison to the SIF stability of oxytocin. The SIF stability of oxytocin is 8±1 min [n.d.: not determined; Ac: acetyl; Cpg: L-cyclopentylglycine; Dpr: L-diaminopropionic acid; (Cα-Me)Leu: α-methyl-L-leucine; Tyr(Me): O-methyl-L-tyrosine; (Cα-Me)Tyr: α-methyl-L-tyrosine; (4-FBzl)Gly: N-(4-fluorobenzyl)glycine; (Cα-Me)Phe: α-methyl-L-phenylalanine; Phe(4-Me): 4-methyl-L-phenylalanine; Phe(4-CF₃): 4-(trifluoromethyl)-L-phenylalanine; Piv: pivaloyl; (Cα-Me)Val: α-methyl-L-valine; Phe(4-F): 4-fluor-L-phenylalanin; Chg: L-cyclohexyl-glycine; thioPro: thioproline; Hyp: L-trans-4-hydroxyproline, (β³-homo)Leu: β³-homoleucine; Lys(γ Glu, α Palm): Lys8 side chain modified with Glu and palmitic acid].

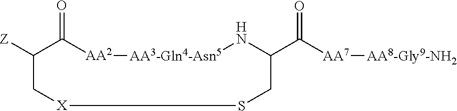

| | | | | | | | SIF | EC₅₀ |
|---|---|---|---|---|---|---|---|---|
| | | | | | | | stability | OTR |
| Compound | Z | X | AA² | AA³ | AA⁷ | AA⁸ | [fold OT] | [nM] |
| OT | NH₂ | S | Tyr | Ile | Pro | Leu | 1 | <10 |
| 1 | NH₂ | S | Tyr | Ile | Pro | Lys(Ac) | 10-100 | <10 |
| 2 | NH₂ | S | Tyr | Ile | Pro | Dpr(Ac) | 10-100 | <10 |
| 3 | NH₂ | S | Tyr | Ile | Pro | (C_{α}-Me)Leu | 10-100 | <10 |
| 4 | NH₂ | S | Tyr | Ile | Pro | (β³-homo)Leu | 10-100 | n.d. |
| 5 | H | CH₂ | Tyr(Me) | Ile | Pro | Lys(Ac) | >100 | <10 |
| 6 | H | CH₂ | Tyr(Me) | Ile | Pro | Dpr(Ac) | >100 | >500 |
| 7 | H | CH₂ | Tyr(Me) | Ile | Pro | (C_{α}-Me)Leu | >100 | 10-500 |
| 8 | H | CH₂ | Tyr(Me) | Ile | (4-FBzl)Gly | Dpr(Ac) | 10-100 | >500 |
| 9 | H | CH₂ | (C_{α}-Me)Tyr | Ile | Pro | Dpr(Ac) | >100 | >500 |
| 10 | H | CH₂ | (C_{α}-Me)Phe | Ile | Pro | Dpr(Ac) | >100 | >500 |
| 11 | H | CH₂ | Phe(4-Me) | Ile | Pro | Dpr(Ac) | 10-100 | 10-500 |
| 12 | H | CH₂ | Phe(4-CF₃) | Ile | Pro | Dpr(Ac) | >100 | >500 |
| 13 | H | CH₂ | Tyr(Me) | Ile | Pro | Dpr(Piv) | > 100 | >500 |
| 14 | H | CH₂ | Tyr(Me) | Cpg | Pro | Dpr(Ac) | >100 | >500 |
| 15 | H | CH₂ | Tyr | (C_{α}-Me)Val | Pro | Dpr(Ac) | >100 | >500 |
| 16 | H | CH₂ | (C_{α}-Me)Tyr | Ile | Pro | Lys(Ac) | >100 | 10-500 |
| 17 | H | CH₂ | (C_{α}-Me)Tyr | Ile | thioPro | Lys(Ac) | >100 | <10 |
| 18 | H | CH₂ | (C_{α}-Me)Tyr | Cpg | thioPro | Lys(Ac) | >100 | <10 |
| 19 | H | CH₂ | Tyr | (C_{α}-Me)Val | Pro | Lys(Ac) | >100 | 10-500 |
| 20 | H | CH₂ | Tyr | (C_{α}-Me)Val | thioPro | Lys(Ac) | > 100 | 10-500 |
| 21 | H | CH₂ | Tyr(Me) | Cpg | Pro | Lys(Ac) | >100 | <10 |
| 22 | H | CH₂ | Tyr(Me) | Ile | thioPro | Lys(Ac) | >100 | <10 |
| 23 | H | CH₂ | Tyr(Me) | Cpg | thioPro | Lys(Ac) | >100 | <10 |
| 24 | H | CH₂ | Phe(4-F) | Ile | thioPro | Lys(Ac) | n.d. | n.d. |
| 25 | H | CH₂ | Tyr | Chg | Hyp | Lys(Ac) | n.d. | n.d. |
| 26 | H | CH₂ | (C_{α}-Me)Tyr | Ile | thioPro | Lys(γGlu,αPalm) | | |

### REFERENCES

[1] Wang, J.; Yadav, V.; Smart, A. L.; Tajiri, S.; Basit, A. W., Toward oral delivery of biopharmaceuticals: an assessment of the gastrointestinal stability of 17 peptide drugs. Molecular Pharmaceutics 2015, 12, (3), 966-73.
[2] Tuppy, H., The Influence of Enzymes on Neurohypophysial Hormones and Similar Peptides. In Neurohypophysial Hormones and Similar Polypeptides, Berde, B., Ed. Springer Berlin Heidelberg: Berlin, Heidelberg, 1968; pp 67-129.
[3] Fjellestad-Paulsen, A.; Soderberg-Ahlm, C.; Lundin, S., Metabolism of vasopressin, oxytocin, and their analogues in the human gastrointestinal tract. Peptides 1995, 16, (6), 1141-1147.
[4] Kremsmayr, T.; Muttenthaler, M., Fmoc Solid Phase Peptide Synthesis of Oxytocin and Analogues. In Oxytocin: Methods and Protocols, Werry, E. L.; Reekie, T. A.; Kassiou, M., Eds. Springer US: New York, NY, 2022; pp 175-199.
[5] Mart, R. J.; Liem, K. P.; Wang, X.; Webb, S. J., The Effect of Receptor Clustering on Vesicle—Vesicle Adhesion. Journal of the American Chemical Society 2006, 128, (45), 14462-14463.
[6] Ichinose, W.; Cherepanov, S. M.; Shabalova, A. A.; Yokoyama, S.; Yuhi, T.; Yamaguchi, H.; Watanabe, A.; Yamamoto, Y.; Okamoto, H.; Horike, S.; Terakawa, J.; Daikoku, T.; Watanabe, M.; Mano, N.; Higashida, H.; Shuto, S., Development of a Highly Potent Analogue and a Long-Acting Analogue of Oxytocin for the Treatment of Social Impairment-Like Behaviors. Journal of Medicinal Chemistry 2019, 62, (7), 3297-3310.
[7] Wisniewski, K.; Alagarsamy, S.; Galyean, R.; Tariga, H.; Thompson, D.; Ly, B.; Wisniewska, H.; Qi, S.; Croston, G.; Laporte, R.; Riviere, P. J.; Schteingart, C. D., New, potent, and selective peptidic oxytocin receptor agonists. Journal of Medicinal Chemistry 2014, 57, (12), 5306-17.
[8] Hicks, C.; Jorgensen, W.; Brown, C.; Fardell, J.; Koehbach, J.; Gruber, C. W.; Kassiou, M.; Hunt, G. E.; McGregor, I. S., The nonpeptide oxytocin receptor agonist WAY 267,464: receptor-binding profile, prosocial effects and distribution of c-Fos expression in adolescent rats. Journal of Neuroendocrinology 2012, 24, (7), 1012-1029.
[9] Keov, P.; Liutkeviciute, Z.; Hellinger, R.; Clark, R. J.; Gruber, C. W., Discovery of peptide probes to modulate oxytocin-type receptors of insects. Scientific Reports 2018, 8, (1), 10020.
[10] Fragiadaki M. et al., Graphical contents list ED. European Journal of Medicinal Chemistry, Elsevier, Amsterdam, NL, Vol 42, No 6, 2007, pages iii-viii, XP025406596, ISSN: 0223-5234
[11] Flouret G. et al. Analogues of a potent oxytocin antagonist with truncated c-terminus or shorter amino acid side chain of the basic amino acid at position 8: oxytocin antagonist analogues. Journal of peptide science, vol. 9, no. 6, 2003, pp. 393-401
[12] Parmar A. Proceedings of the World Molecular Imaging Congress 2019, Montreal Quebec, Canada Sep. 4-7, 2019: Late-Breaking Abstracts. Molecular Imaging & Biology, Elsevier, Boston, vol. 21, no. Suppl 1, 2019, pp. 167-335, XP037077346, ISSN: 1536-1632
[13] Muttenthaler M. et al., Modulating Oxytocin Activity and Plasma Stability by Disulfide Bon Engineering. Journal of medicinal chemistry, vol. 53, no. 24, 2010, pp. 8585-8596
[14] Dongren R. et al., Genetic Diversity in Oxytocin Ligands and Receptors in New World Monkeys. PLOS ONE, vol. 10, no. 5, 2015

1. A compound of general formula I 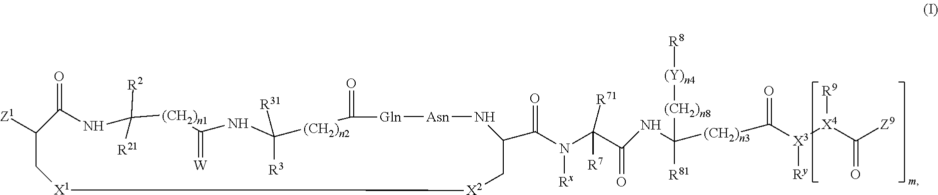
wherein
Z¹ (N-terminus) denotes NH₂, H, or OH;
Z⁹ (C-terminus) denotes NH₂, or OH; or
Z⁹ denotes NH if m denotes 2, 3, 4, 5, or 6;
X¹ and X² independently from one another denote S, Se, or CH₂;
R²¹, R³¹, R⁷¹, and R⁸¹ independently from one another denote H, C₁₋₆alkyl, halogen, or CF₃;
R^{x} denotes H or a group, optionally substituted by C₆₋₁₀ aryl and/or optionally substituted by one or more identical or different R^{a} and/or R^{b}, selected from among C₁₋₆ alkyl, C₂₋₆ alkenyl, or C₂₋₆ alkynyl;
R^{y} denotes H or a group, optionally substituted by C₆₋₁₀ aryl and/or optionally substituted by one or more identical or different R^{a} and/or R^{b}, selected from C₁₋₆alkyl, C₂₋₆alkenyl, or C₂₋₆alkynyl;
n1 denotes 0, 1, 2, or 3, and wherein n1 denotes 0 if R²¹ is different from H;
n2 denotes 0, 1, 2, or 3, and wherein n2 denotes 0 if R³¹ is different from H;
n3 denotes 0, 1, 2, or 3, and wherein n3 denotes 0 if R⁸¹ is different from H;
n4 denotes 0 or 1;
n8 denotes 0, 1, 2, 3, 4, 5, 6, 7, 8, 9, or 10;
m denotes 0, 1, 2, 3, 4, 5, or 6;
W denotes O or S;
X³ denotes N; or
X³ denotes O or NH if m denotes 0;
X⁴ denotes CH or N;
Y denotes NH or O;
R² denotes Tyr together with the peptide backbone of residue 2; or
R² denotes H or a group, optionally substituted by one or more identical or different R^{a} and/or R^{b}, selected independently from among C₁₋₁₀ alkyl, C₂₋₁₀ alkenyl, C₂₋₁₀ alkynyl, C₃₋₈ cycloalkyl, C₄₋₈ cycloalkylalkyl, C₆₋₈ aryl, C₇₋₈ arylalkyl, 3-8 membered heterocycloalkyl, 4-8 membered heterocycloalkylalkyl, 5-8 membered heteroaryl, and 6-8 membered heteroarylalkyl;
R³ denotes Ile together with the peptide backbone of residue 3; or
R³ denotes H or a group, optionally substituted by one or more identical or different R^{a} and/or R^{b}, selected independently from among C₁₋₁₀ alkyl, C₂₋₁₀ alkenyl, C₂₋₁₀ alkynyl, C₃₋₈ cycloalkyl, C₄₋₈ cycloalkylalkyl, C₆₋₈ aryl, C₇₋₈ arylalkyl, 3-8 membered heterocycloalkyl, 4-8 membered heterocycloalkylalkyl, 5-8 membered heteroaryl, and 6-8 membered heteroarylalkyl;
R⁷ denotes H or a group, optionally substituted by one or more identical or different R^{a} and/or R^{b}, selected independently from among C₁₋₁₀ alkyl, C₂₋₁₀ alkenyl, C₂₋₁₀ alkynyl, C₃₋₈ cycloalkyl, C₄₋₈ cycloalkylalkyl, C₆₋₈ aryl, C₇₋₈ arylalkyl, 3-8 membered heterocycloalkyl, 4-8 membered heterocycloalkylalkyl, 5-8 membered heteroaryl, and 6-8 membered heteroarylalkyl; or
R⁷ together with R^{x} forms an optionally substituted 3 to 6 membered heterocycloalkyl ring;
R⁸ denotes —C(O)R^{c}, —C(R^{c})═NOR^{c}, or a group, optionally substituted by one or more identical or different R^{a} and/or R^{b}, selected independently from among C₁₋₁₀ alkyl, C₂₋₁₀ alkenyl, C₂₋₁₀ alkynyl, 3-13 membered heterocycloalkyl, and 5-13 membered heteroaryl, and wherein R⁸ optionally comprises a terminal lipidation and/or PEGylation, and/or labeling;
R⁹ denotes H or C₁₋₅alkyl;
each R^{a} independently of one another denotes H or a group, optionally substituted by one or more identical or different R^{b} and/or R^{c}, selected from among C₁₋₆alkyl, C₂₋₆alkenyl, C₂₋₆alkynyl, C₃₋₈cycloalkyl, C₄₋₈cycloalkylalkyl, C₆₋₈ aryl, C₇₋₈ arylalkyl, 3-8 membered heterocycloalkyl, 4-8 membered heterocycloalkylalkyl, 5-8 membered heteroaryl, and 6-8 membered heteroarylalkyl;
each R^{b} is a suitable substituent and is selected in each case independently of one another from among ═O, —OR^{c}, C₁₋₃haloalkyloxy, —OCF₃, ═S, —SR^{c}, =NR^{c}, =NOR^{c}, —NR^{c}R^{c}, —ONR^{c}R^{c}, —N(OR^{c})R^{c}, —N(R^{g})NR^{c}R^{c}, halogen, —CF₃, —CN, —NC, —OCN, —SCN, —NO, —NO₂, =N₂, —N₃, —S(O)R^{c}, —S(O)OR^{c}, —S(O)₂R^{c}, —S(O)₂OR^{c}, —S(O)NR^{c}R^{c}, —S(O)₂NR^{c}R^{c}, —OS(O)R^{c}, —OS(O)₂R^{c}, —OS(O)₂OR^{c}, —OS(O)NR^{c}R^{c}, —OS(O)₂NR^{c}R^{c}, —C(O)R^{c}, —C(O)OR^{c}, —C(O)SR^{c}, —C(O)NR^{c}R^{c}, —C(O)N(R^{g})NR^{c}R^{c}, —C(O)N(R^{g})OR^{c}, —C(NR^{g})NR^{c}R^{c}, —C(NOH)R^{c}, —C(NOH)NR^{c}R^{c}, —OC(O)R^{c}, —OC(O)OR^{c}, —OC(O)SR^{c}, —OC(O)NR^{c}R^{c}, —OC(NR^{g})NR^{c}R^{c}, —SC(O)R^{c}, —SC(O)OR^{c}, —SC(O)NR^{c}R^{c}, —SC(NR⁹)NR^{c}R^{c}, —N(R^{g})C(O)R^{c}, and —N[C(O)R^{c}]₂;
each R^{e} independently of one another denotes H or a group, optionally substituted by one or more identical or different R^{d} and/or R^{e}, selected from among C₁₋₆alkyl, C₂₋₆alkenyl, C₂₋₆ alkynyl, C₃₋₈ cycloalkyl, C₄₋₈ cycloalkylalkyl, C₆₋₈ aryl, C₇₋₈ arylalkyl, 3-8 membered heterocycloalkyl, 4-8 membered heterocycloalkylalkyl, 5-8 membered heteroaryl and 6-8 membered heteroarylalkyl;
each R^{d} denotes a suitable substituent and is selected in each case independently of one another from among ═O, —OR^{e}, C₁₋₃haloalkyloxy, —OCF₃, ═S, —SR^{e}, =NR^{e}, =NOR^{e}, —NR^{e}R^{e}, —ONR^{e}R^{e}, —N(OR^{e})R^{e}, —N(R^{g})NR^{e}R^{e}, halogen, —CF₃, —CN, —NC, —OCN, —SCN, —NO, —NO₂, =N₂, —N₃, —S(O)R^{e}, —S(O)OR^{e}, —S(O)₂R^{e}, —S(O)₂OR^{e}, —S(O)NR^{e}R^{e}, —S(O)₂NR^{e}R^{e}, —OS(O)R^{e}, —OS(O)₂R^{e}, —OS(O)₂OR^{e}, —OS(O)NR^{e}R^{e}, —OS(O)₂NR^{e}R^{e}, —C(O)R^{e}, —C(O)OR^{e}, —C(O)SR^{e}, —C(O)NR^{e}R^{e}, —C(O)N(R^{g})NR^{c}R^{c}, —C(O)N(R^{g})OR^{e}, —C(NR^{g})NR^{e}R^{e}, —C(NOH)R^{e}, —C(NOH)NR^{e}R^{e}, —OC(O)R^{e}, —OC(O)OR^{e}, —OC(O)SR^{e}, —OC(O)NR^{e}R^{e}, —OC(NR^{g})NR^{c}R^{c}, —SC(O)R^{e}, —SC(O)OR^{e}, —SC(O)NR^{e}R^{e}, —SC(NR⁹)NR^{e}R^{e}, —N(R^{g})C(O)R^{e}, and —N[C(O)R^{e}]₂; and
each R^{e} independently of one another denotes H or a group selected from among C₁₋₆alkyl, C₂₋₆alkenyl, C₂₋₆alkynyl, C₃₋₈ cycloalkyl, C₆₋₈ aryl, 3-8 membered heterocycloalkyl, and 5-8 membered heteroaryl; and
each R^{g} independently of one another denotes H, C₁₋₆alkyl, C₂₋₆alkenyl, C₂₋₆alkynyl, C₃₋₈ cycloalkyl, C₆₋₈ aryl, 3-8 membered heterocycloalkyl, or 5-8 membered heteroaryl;
with the proviso that R⁸, n4, n8, and Y together with the peptide backbone of residue 8 is different from Leu, Ile, cyclopentyl-Gly (Cpg), Lys, Arg, or the enantiomers thereof;
with the proviso that if R⁸ comprises a terminal lipidation and/or PEGylation and/or labeling, R² together with the peptide backbone of residue 2 is different from Tyr, and/or R³ together with the peptide backbone of residue 3 is different from Ile, and/or one of X¹ or X² is different from S; and
with the proviso that if R⁸, n4, n8, and Y together with the peptide backbone of residue 8 is Ala or Thr, then R² together with the peptide backbone of residue 2 is different from Tyr, and/or R³ together with the peptide backbone of residue 3 is different from Ile, and/or one of X¹ or X² is different from S;
optionally in the form of the tautomers, the racemates, the enantiomers, the diastereomers, hydrates, and mixtures thereof, and optionally the pharmacologically acceptable salts thereof.

2. The compound of claim 1, wherein R⁸ is —C(O)R^{c}, —C(R′)=NOR^{c}, or a group, optionally substituted by one or more identical or different R^{a} and/or R^{b}, selected independently from among C₁₋₁₀alkyl, C₂₋₁₀ alkenyl, C₂₋₁₀ alkynyl, 3-13 membered heterocycloalkyl, or 5-13 membered heteroaryl, wherein R^{b} is preferably selected from among ═O, —OR^{c}, C₁₋₃haloalkyloxy, —OCF₃, —NR^{c}R^{c}, —N(OR^{c})R^{c}, halogen, —CF₃, —C(O)R^{c}, —C(O)OR^{c}, —C(O)NR^{c}R^{c}, —C(O)N(R^{g})NR^{c}R^{c}, —C(O)N(R^{g})OR^{c}, —C(NR^{g})NR^{c}R^{c}, and —N(R^{g})C(O)R^{c}.

3. The compound of claim 1, wherein the terminal lipidation and/or PEGylation of R⁸ comprises a group selected from the group consisting of polyethylene glycol (PEG), PEG substituted with one or more amino acids and/or one or more fatty acids, and (PEG substituted with one amino acid)1-5 optionally substituted with one or more fatty acid.

4. (canceled)

5. (canceled)

6. (canceled)

7. The compound of claim 1, wherein the terminal labeling of R⁸ comprises a group selected from the group consisting of biotin; fluorophores; metal chelators, preferably DOTA, NODA and NODAGA; antigens; MS-, NMR-, PET- and MRI-active tags; technical recognition tags; and biological recognition tags.

8. The compound of claim 1, wherein R² denotes Tyr together with the peptide backbone of residue 2, or a C₁₋₁₀alkyl group optionally substituted by R^{a}, preferably by C₆₋₈ aryl, C₇₋₈arylalkyl, 5-8 membered heteroaryl, and 6-8 membered heteroarylalkyl, optionally further substituted by one or more identical or different R^{b} and/or R^{c}, preferably by C₁₋₆alkyl, ═O, —OR^{c}, C₁₋₃haloalkyloxy, —OCF₃, —NR^{c}R^{c}, —N(OR^{c})R^{c}, halogen, —CF₃, —C(O)R^{c}, —C(O)OR^{c}, —C(O)NR^{c}R^{c}, —C(O)N(R^{g})NR^{c}R^{c}, —C(O)N(R^{g})OR^{c}, —C(NR⁹)NR^{c}R^{c}, or —N(R^{g})C(O)R^{c}.

9. The compound of claim 1, wherein R³ denotes Ile together with the peptide backbone of residue 3, or a group, selected independently from among C₁₋₁₀alkyl, C₃₋₈cycloalkyl, optionally substituted by one or more identical or different R^{a} and/or R^{b}, wherein R^{b} is preferably selected from among ═O, —OR^{c}, C₁₋₃halo-alkyloxy, —OCF₃, —NR^{c}R^{c}, —N(OR^{c})R^{c}, halogen, —CF₃, —C(O)R^{c}, —C(O)OR^{c}, —C(O)NR^{c}R^{c}, —C(O)N(R^{g})NR^{c}R^{c}, —C(O)N(R^{g})OR^{c}, —C(NR⁹)NR^{c}R^{c}, and —N(R^{g})C(O)R^{c}.

10. The compound of claim 1, wherein R⁷ denotes H or C₁₋₁₀ alkyl optionally substituted by one or more identical or different R^{a} and/or R^{b}, wherein R^{b} is preferably selected from among ═O, —OR^{c}, C₁₋₃haloalkyloxy, —OCF₃, —NR^{c}R^{c}, —N(OR^{c})R^{c}, halogen, —CF₃, —C(O)R^{c}, —C(O)OR^{c}, —C(O)NR^{c}R^{c}, —C(O)N(R^{g})NR^{c}R^{c}, —C(O)N(R^{g})OR^{c}, —C(NR⁹)NR^{c}R^{c}, and —N(R^{g})C(O)R^{c}, or R⁷ together with R^{x} forms an optionally substituted 3 to 5 membered heterocycloalkyl ring, preferably pyrrolidine, thiazolidine, or hydroxypyrrolidine.

11. The compound of any claim 1, wherein R²¹, R³¹, R⁷¹, and R⁸¹ independently from one another denote H, C₁₋₆alkyl, halogen, or CF₃.

12. The compound of claim 1, wherein R²¹ denotes H or methyl; R³¹ denotes H or methyl; R⁷¹ denotes H; and/or R⁸¹ denotes H or methyl.

13. (canceled)

14. (canceled)

15. (canceled)

16. The compound of claim 1, wherein R² denotes a C₁₋₃alkyl group substituted by optionally substituted C₆₋₈ aryl.

17. (canceled)

18. The compound of claim 1, wherein R³ denotes Ile together with the peptide backbone of residue 3.

19. The compound of claim 1, wherein R³ denotes optionally substituted C₁₋₁₀alkyl or C₅₋₆cycloalkyl.

20. The compound of claim 1, wherein R⁷ denotes H or R⁷ together with R^{x} forms an optionally substituted 3 to 5 membered heterocycloalkyl ring.

21. (canceled)

22. The compound of claim 1, wherein R^{x} denotes H or optionally substituted C₁₋₆alkyl.

23. The compound of claim 1, wherein Z⁹, R^{y}, m, X³, X⁴, and R⁹ together form Gly-NH₂.

24. A compound of general formula I according to claim 1, wherein the compounds are represented by formula VI, 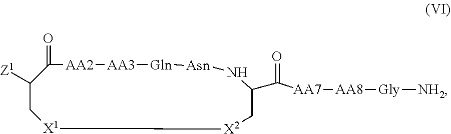
wherein
Z¹ denotes NH₂ or H;
X¹ and X² independently from one another denote S or CH₂;
AA2 denotes Tyr, Tyr(Me), (Ca-Me)Tyr, (Ca-Me)Phe, Phe(4-Me), Phe(4-CF₃), or Phe(4-F);
AA3 denotes Ile, Cpg, (Ca-Me)Val, or Chg;
AA7 denotes Pro, (4-FBzl)Gly, thioPro, or Hyp;
AA8 denotes Lys(Ac), Dpr(Ac), (Ca-Me)Leu, (β³-homo)Leu, Dpr(Piv), or Lys(γGlu,αPalm).

25. A method of treating a disease in a patient in need thereof, the method comprising administering to the patient a compound of general formula I according to claim 1, optionally in the form of the tautomers, the racemates, the enantiomers, the diastereoisomers, hydrates, and mixtures thereof, or the pharmaceutically acceptable salts thereof.

26. A method of treating or preventing gastrointestinal disorders, the method comprising administering to a subject in need thereof a compound of general formula I according to claim 1, optionally in the form of the tautomers, the racemates, the enantiomers, the diastereoisomers, hydrates, and mixtures thereof, or the pharmaceutically acceptable salts thereof, wherein the gastrointestinal disorders include symptoms and/or causes associated with inflammatory bowel diseases (IBD) including ulcerative colitis and Crohn's disease and symptoms and/or causes associated with irritable bowel syndrome (IBS), wherein said symptoms and/or causes are abdominal/visceral pain, constipation, diarrhea, abnormal bowel movement, inflammation and rectal bleeding.

27. Pharmaceutical preparation containing as active substance one or more compounds of general formula I according to claim 1, optionally in the form of the tautomers, the racemates, the enantiomers, the diastereoisomers, hydrates, and mixtures thereof, or the pharmaceutically acceptable salts thereof, optionally in combination with conventional excipients and/or carriers.

28. Pharmaceutical preparation comprising a compound of general formula I according to a claim 1, optionally in the form of the tautomers, the racemates, the enantiomers, the diastereoisomers, hydrates, and mixtures thereof, or also pharmaceutically acceptable salts of all the above-mentioned forms, and at least one further active substance different from formula I.


SEQUENCE LISTING (1 sequence)

SEQ ID NO: 1 - protein, 9 aa, unidentified
  VARIANT 1..9: note = Mammalia
CYIQNCPLG